(19) United States
(12) Patent Application Publication
(10) Pub. No.: US 2026/0260703 A1
(43) Pub. Date: Sep. 3, 2026

(54) DEVICES AND METHODS INVOLVING ANALYSIS OF PATIENT DATA BASED ON NUCLEIC ACID SEQUENCE ANALYSIS

(71) Applicant: FRED HUTCHINSON CANCER CENTER, Seattle, WA (US)

(72) Inventors: Eric Holland, Seattle, WA (US); Matt Jensen, Seattle, WA (US); Nicholas Nuechterlein, Seattle, WA (US); Sonali Arora, Alpharetta, GA (US)

(21) Appl. No.: 19/447,743

(22) Filed: Jan. 13, 2026

Related U.S. Application Data
(63) Continuation of application No. PCT/US2024/054435, filed on Nov. 4, 2024.
(60) Provisional application No. 63/595,717, filed on Nov. 2, 2023.
(60) Provisional application No. 63/702,539, filed on Oct. 2, 2024.

(51) Int. Cl. G16B 25/10; G16B 40/30; G16H 10/60; G16H 20/10; G16H 50/70

(52) U.S. Cl. CPC G16B 25/10; G16B 40/30; G16H 10/60; G16H 20/10; G16H 50/70

(57) ABSTRACT

In certain examples, computer-implemented methods involve assessing patient diagnosis utilizing RNA expression profiles. As may be implemented in accordance with one or more aspects characterized herein, a landscape of RNA expression profiles may be generated from a plurality of patients having a set of common medical attributes. An RNA expression profile is obtained for a target patient having the medical attributes, and a plurality of nearest neighbor RNA expression profiles are identified from the landscape of RNA expression profiles, based on the target patient's RNA expression profile. A diagnosis for the target patient is provided, or identification of certain ones of the patients is identified, based on known characteristics of the plurality of patients corresponding to the identified nearest neighbor RNA expression profiles.

### BACKGROUND

Aspects of the present disclosure are related generally to the field of nucleic acid sequence analysis, and as may be exemplified by uses in assessing patients for diagnosing medical conditions.

With regard to cancer, addressing the cancer may involve observing, predicting, or otherwise determining how changes in genes or proteins in the cancer cells of a patient might affect the patient's care, such as the pharmaceuticals being prescribed or treatment being provided. Generally, a healthcare professional—for example, an oncologist—uses information obtained or derived from laboratory tests to develop a personalized plan of care that includes recommendations tailored for the patient. This information could be used by the healthcare professional to inform of diagnoses or treatment. This information could also be used to indicate when screening is needed, whether the patient is at higher risk for a given cancer, or whether treatment is working as intended. Simply put, this information can be used in various ways to improve the care—and, therefore, the outcomes—of patients.

Historically, such approaches have been largely based on knowing the effects of changes in genes (and proteins) inside cells. Genes are pieces of deoxyribonucleic acid (“DNA”) inside each cell. At a high level, genes are representative of the instructions that indicate, to the cell, how to make the proteins that are needed to properly function. Each gene contains instructions to make a certain protein, and each protein has a certain job in the cell.

All cancers are caused by gene changes of some kind. Cancer cells are abnormal versions of normal cells, meaning that something changes in the genes of that normal cell to turn it into a cancer cell. For example, genes that normally help keep cells from growing out of control might be “turned off,” or genes that normally help cells grow and divide might be “turned on” all the time. Significant improvements have been made in discovering these gene changes. However, it is still difficult for healthcare professionals—even seasoned ones—to appropriately personalize the care of a patient based on gene changes.

These and other aspects have presented challenges to diagnosing medical conditions such as cancer.

### SUMMARY OF VARIOUS ASPECTS AND EXAMPLES

Various examples/embodiments presented by the present disclosure are directed to issues such as those addressed above and/or others which may become apparent from the following disclosure. For example, some of these disclosed aspects are directed to methods and devices that use or leverage from analysis of RNA expression profiles from several patients, and landing a new patient on one or several of the RNA expression profiles for assessing similar characteristics (e.g., for providing a similar diagnosis). Other aspects are directed to enhancing previously-used techniques, such as discussed above, by providing a manner in which to generate and output diagnoses with learned historical diagnoses.

In one specific example, a method involves generating a “landscape” of RNA expression profiles from a plurality of patients having a set of medical attributes that is common to the patients. The term “landscape” may refer to a visual representation of biological data, such as RNA expression profiles from a group of patients in a particular biomedical context, or a related representation of data points that may or may not be presented in a visual form. For a target patient having the general medical diagnosis, an RNA expression profile is ascertained from the target patient, for example as a tumor sample or from normal tissue in a control group of “normal” subjects. A plurality of nearest neighbor RNA expression profiles are identified from the landscape of RNA expression profiles, based on the target patient's RNA expression profile, including computing a distance (or a set of one or more distances) corresponding to one or more centroid values pertaining to the patients. A diagnosis for the target patient's medical condition is generated and made available (e.g., presented as an output) based on known characteristics of the plurality of patients corresponding to the identified nearest neighbor RNA expression profiles.

In certain other examples that may also build on the above-discussed aspects, a landscape of RNA expression profiles is generated by arranging RNA sequence (“RNA seq”) data from the plurality of patients in a lower dimensional representation, and identifying the plurality of nearest neighbor RNA expression profiles as follows. A visualization that includes a plurality of visual indicia for the plurality of patients is obtained and a list is compiled to include each existing patient of the plurality of patients that are established as nearest neighbors. Coordinates within the lower dimensional representation for the target patient are determined and, based on the coordinates, one or more of the plurality of patients are established as nearest neighbors to the target patient in the lower dimensional representation.

In a further specific example as may also build on the above-discussed aspects, a centroid value may be identified in the visualization for the target patient as follows. A centroid of values corresponding to the patients in the list is calculated, and a distance from the centroid to the values corresponding to each existing patient of the plurality of patients is computed. The values corresponding to the plurality of patients are filtered based on the computed distance for each of the plurality of patients and a predetermined percentile in terms of the computed distance from the centroid, therein producing filtered values. The centroid is recalculated for the filtered values and the filtered values are further filtered based on a radius of the recalculated centroid, therein producing a remaining set of filtered values. A centroid is calculated as an approximate location for the target patient based on coordinates of the patients corresponding to the remaining set of filtered values.

Various aspects, as may be implemented in accordance with the above and/or otherwise, a statistically robust, dimensionality-reduced landscape is created for a large number of entities, each represented as a collection of data. Clusters in the landscape may be utilized to provide an understanding as to underlying reasons as to why such clusters exist. The resulting landscape can be utilized to accurately land a new entity in one of the clusters, and to infer properties of the new entity relative to the cluster in which the entity has landed.

Another embodiment is directed to a computer-implemented method comprising generating, from a plurality of patients (“the patients”), a landscape of RNA expression profiles having a set of one or more medical attributes that is common to the patients. For a target patient having the set of medical attributes, an RNA expression profile of the target patient is ascertained and a plurality of nearest neighbor RNA expression profiles are identified from the landscape of RNA expression profiles, based on the target patient's RNA expression profile and at least one computed distance corresponding to at least one centroid value for the RNA expression profiles. Data for a diagnosis or other health-specific recommendation are generated and outputted for the target patient based on known characteristics of the patients corresponding to the identified plurality of nearest neighbor RNA expression profiles.

Another embodiment is directed to an apparatus comprising processing circuitry to generate a landscape of RNA expression profiles from a plurality of patients (“the patients”) having a set of medical attributes that is common to the patients. For a target patient having the set of medical attributes, the processing circuitry is to ascertain the target patient's RNA expression profile and identify a plurality of nearest neighbor RNA expression profiles from the landscape of RNA expression profiles based on the target patient's RNA expression profile, including computing a distance corresponding to one or more centroid values pertaining to RNA expression profile values for the patients. Data for a diagnosis for the target patient is generated and outputted based on known characteristics of the patients corresponding to the identified nearest neighbor RNA expression profiles.

The above discussion is not intended to describe each aspect, embodiment or every implementation of the present disclosure. The figures and detailed description that follow also exemplify various embodiments.

### BRIEF DESCRIPTION OF FIGURES

Various example embodiments, including experimental examples, may be more completely understood in consideration of the following detailed description in connection with the accompanying drawings, each in accordance with the present disclosure, in which:

FIG. 1 includes a flow diagram of a workflow for generating a visualization based on RNA seq data, according to certain exemplary aspects of the present disclosure;

FIGS. 2A and 2B show a flow diagram of a process for overlaying a patient newly diagnosed with a disease (e.g., meningioma) on a visualization, so as to visually illustrate the relationship with a plurality of existing patients known to have the disease, according to certain exemplary aspects of the present disclosure;

FIG. 3 includes a flow diagram of a process for improving classification of a patient newly diagnosed with a disease (e.g., meningioma), according to certain exemplary aspects of the present disclosure;

FIG. 4 includes a flow diagram of a process for identifying a cohort of nearest neighbors and producing an appropriate output, according to certain exemplary aspects of the present disclosure;

FIG. 5 includes a flow diagram of a process for comparing two selected cohorts of patients, for example, through subtraction of an average value versus another average value or differential gene expression, according to certain exemplary aspects of the present disclosure;

FIG. 6 illustrates a network environment that includes a disease analysis platform that is executed by a computing device, according to certain exemplary aspects of the present disclosure;

FIG. 7 illustrates how the disease analysis platform may be accessible to different types of users via corresponding portals, according to certain exemplary aspects of the present disclosure;

FIG. 8 illustrates an example of a computing device that is able to implement a disease analysis platform designed to produce visualizations based on RNA seq data, according to certain exemplary aspects of the present disclosure; and

FIG. 9 includes a block diagram illustrating an example of a processing system in which at least some operations described herein can be implemented, according to certain exemplary aspects of the present disclosure.

While various embodiments discussed herein are amenable to modifications and alternative forms, aspects thereof have been shown by way of example in the drawings and will be described in detail. It should be understood, however, that the intention is not to limit the disclosure to the particular embodiments described. On the contrary, the intention is to cover all modifications, equivalents, and alternatives falling within the scope of the disclosure including aspects defined in the claims. In addition, the term “example” as used throughout this application is only by way of illustration, and not limitation.

### DETAILED DESCRIPTION

Aspects of the present disclosure are believed to be applicable to a variety of different types of apparatuses, systems and methods involving devices characterized at least in part by the assessment of medical conditions and as may relate to diagnoses of such conditions, for example as relative to cancer. While the present disclosure is not necessarily limited to such aspects, an understanding of specific examples in the following description may be understood from discussion in such specific contexts.

Gene changes can affect not only how a cancer responds to treatment, but also decisions on what, if any, treatment is appropriate. For instance, tumors that form in the meninges—commonly called “meningiomas,” may or may not be cancerous. Treatment of non-cancerous meningiomas may not be necessary, or at least urgent. However, if a meningioma is cancerous, it is aggressive and able to invade other tissues, potentially spreading to other parts of the body. Accordingly, it is important for healthcare professionals to quickly establish whether a meningioma is cancerous so that appropriate personalized care can be provided.

Differences between patients in terms of gene changes in their cancers has historically made this difficult. Even for the same type of cancer, some patients will have gene changes that are different from those in other patients. As such, a “one-size-fits-all” approach is not appropriate for prescribing treatment. However, the assessment of patients has been limited by the inability to gain insights into the relationships between patients, even those with the same type of cancer. Accordingly, various aspects of the present disclosure are directed to approaches to programmatically establishing relationships between patients known to have a given disease, such that personalized care more likely to lead to desired outcomes can be provided. As further discussed herein and also according to aspects of the present disclosure, these relationships may be visually illustrated through the production of visual representations (also called “visualizations”), and such visualizations may be displayed for view by an individual (e.g., via a graphic user interface as may be provided via a computer) and/or may be characterized by processing and/or generating output data via a computer-implemented algorithm; analyses (e.g., computer-implemented) of such visualizations are used to generate insights into appropriate diagnoses, treatments, and other health-related recommendations (e.g., adjustment of prescriptions, surgery, therapy, etc.). In the specific context of cancer-related applications, these insights allow for precision oncology, as information known about existing patients, which can be used to better serve patients that have been newly diagnosed with the given disease. A patient in this regard may refer to a being who may be susceptible to a condition or diagnosis whether or not treatment is sought or necessary.

As further discussed below, various approaches can be implemented by a disease (computer-implemented) analysis platform (or simply “analysis platform”). In operation, the analysis platform can implement a framework for gaining insights into a given disease and its underlying biology from an analysis of nucleic acid sequences. Assume, for example, that the analysis platform obtains a dataset that includes sequence information (e.g., ribonucleic acid sequences) for samples taken from neoplastic brain tissue associated with individuals that are known to have brain tumors and samples taken from normal brain tissue (also called “healthy brain tissue”) associated with individuals that are presumed not to have brain tumors. Collectively, these neoplastic individuals and healthy individuals may be called the “cohort” or “set” of individuals whose data is included in the dataset. The analysis platform can then normalize the sequence information—for example, to transcripts per million (“TPM”)—and perform batch correction.

Thereafter, the analysis platform can construct a visualization of the sequence information. Such a visualization may be obtained by a computer categorizing and assessing the data, and may or not produce a visible characterization of the corresponding data. Each individual whose information is included in the sequence information may be represented, in the visualization, by a different visual indicium. For example, the analysis platform may fit a Uniform Manifold Approximation and Projection (“UMAP”) model to the sequence information. UMAP is commonly used for visualization by reducing higher-dimension data to two dimensions, for example, in the form of a scatter plot where the points are representative of the individuals in the cohort. Other types of data can then be overlaid on the visualization by the analysis platform. This data may include genomic information, as well as information related to the given disease (e.g., severity, date of diagnosis, etc.), treatment (e.g., prescribed medications, procedures), services (e.g., location of healthcare facility, name of healthcare provider), and the like.

Accordingly, in the following description various specific details are set forth to describe specific examples presented herein. It should be apparent to one skilled in the art, however, that one or more other examples and/or variations of these examples may be practiced without all the specific details given below. In other instances, well known features have not been described in detail so as not to obscure the description of the examples herein. For ease of illustration, the same connotation and/or reference numerals may be used in different diagrams to refer to the same elements or additional instances of the same element. Also, although aspects and features may in some cases be described in individual figures, it will be appreciated that features from one figure or embodiment can be combined with features of another figure or embodiment even though the combination is not explicitly shown or explicitly described as a combination.

Consistent with the above aspects, a manufactured device, a method of such manufacture, and approaches for assessing patient diagnoses may involve aspects presented and claimed in U.S. Provisional Application Ser. No. 63/595,717 (“the '717 Provisional”) filed on Nov. 2, 2023(070354.8009.US00) including Appendices that form part of the Provisional Application, and to U.S. Provisional Application Ser. No. 63/702,539 (“the '539 Provisional”) filed on Oct. 2, 2024 (FHCC.1 23-222-US-PSP2), to which priority is claimed. To the extent permitted, such subject matter is incorporated by reference in its entirety generally and to the extent that further aspects and examples (such as experimental and/more-detailed embodiments) may be useful to supplement and/or clarify.

Consistent with the present disclosure, such methods and/or apparatuses may be used for producing (among other examples disclosed herein) a detailed diagnosis for a patient, relative to a landscape of prior patients having a common medical diagnosis. Such a detailed diagnosis may, for example, provide more specific predictive diagnostic characteristics relative to general diagnosis pertaining to the larger group of patients. For instance, a statistically robust, dimensionality-reduced landscape may be created for a large number of patients, each represented as a collection of data, studying clusters in the landscape to understand the underlying reasons they exist, and using that landscape to accurately land a new patient and make inferences relative to the region in which the patient has landed. These inferences may include diagnosis of disease, disease-related prognosis, or risk of developing disease.

In a more particular example, such an approach may involve identifying a patient's clinically-related closest cohort of other patients for purposes of diagnosis and treatment optimization. A statistically-robust, dimensionally-reduced landscape of RNA expression profiles are created from many patients'tumors using tools such as t-SNE (t-Distributed Stochastic Neighbor Embedding), UMAP (Uniform Manifold Approximation and Projection), and TDA (Topological Data Analysis), which may be customized. A new/target patient is landed via the patient's RNA expression profile on that landscape with statistical robustness, which may facilitate ascertaining molecular characteristics, lifespan, optimal therapy and other traits for the new/target patient based on the neighbors in the landscape. This may involve identifying nearest neighbors as ones of the patients'in the reference landscape that are most similar to the new/target patient being landed. The nearest neighbors can be utilized to collectively predict the behavior of the new/target patient being landed. Various such aspects may involve machine learning (ML)/artificial intelligence (AI) meta layers.

Certain embodiments are directed to a method of predicting risk for a user using their electronic medical record information. This may involve creating a statistically-robust, dimensionally-reduced landscape of electronic medical records from many patients using existing tools as noted above. A new/target patient's electronic medical record may be landed on that landscape and utilized for assessing risk of developing various diseases (“risk profile”) and other traits for the new/target patient based on the neighborhood they land in. As such, the most similar patients in the reference landscape may be utilized as a best guess as to the behavior of the new/target patient being landed. Skilled humans may understand the clusters of patients that typically form in the landscape, and/or machine learning/AI meta layers could also do that and distil information in forms humans could understand.

As discussed herein and consistent with the above-referenced provisional applications, an analysis platform can employ a more consistent, programmatic approach to predicting diagnoses, outcomes, and responses to treatments for patients having diseases of all types. Specifically, the analysis platform can be used:
As a diagnostic tool. For example, the analysis platform may generate an interface on which multiple cancers are placed on the same UMAP model, either with or without normal samples corresponding to individuals that are determined to not have any of the multiple cancers. With this UMAP model, the analysis platform can show that (i) there are different cancers located in different parts of the UMAP model; (ii) there are diagnostic errors that have been made by healthcare professionals, which are discovered via analysis of the marks corresponding to individual patients—demonstrating superiority to standard pathology; and (iii) there are regions of the UMAP model that correspond, mostly or entirely, to particular types of cancer. Moreover, analysis of the UMAP model may lead to insights such as desirable outcomes (e.g., survival) tend to correlate to a cluster of patients within a region—and therefore, that location within the UMAP model may be predictive of outcome for a given type of cancer. Moreover, coloring in the UMAP model may be based on metadata as discussed above, and this metadata—or analyses thereof—may provide biological insight into the different types of cancer shown in the UMAP model.
As a mechanism for subdividing tumor diagnosis. Multiple datasets could be combined to generate a useful UMAP model as discussed above. These multiple datasets could be combined into a single dataset to generate a useful UMAP model. It has been shown that the meningiomas fall into nine different clusters with distinct outcomes, as discussed above. Moreover, the analysis platform can show that the biology of each cluster (or subcluster) is different. While the details may be specific to the type of cancer under consideration (here, meningiomas), the ability to readily understand the biology of tumor subsets allows for greater insight into the most effective treatments. Such an approach can greatly improve upon existing techniques for grading cancer, as grading (e.g., WHO Grade I, Grade II, or Grade III) tends to lose specificity that is necessary for the development of personalized treatment plans. Establishing the nearest neighbors for a patient, rather than just patients having the same grade, allows for greater predictability of treatment success.

Accordingly, the analysis platform can serve as a diagnostic assistance tool. For instance, the analysis platform may predict, determine and/or inform diagnoses, for example depending on supporting data that may be sufficient so as to override the need to predict, and/or may focus on performing actions (e.g., identifying nearest neighbors and presenting information regarding treatments or outcomes of those nearest neighbors) that help healthcare professionals diagnose patients. Referring to the analysis platform itself, there are several aspects that empower a user to make observations. Features of the analysis platform include but are not limited to:
The ability to visualize a dataset that includes information for multiple patients, for example, as a UMAP model or another visualization;
The ability to calculate various dimension-reduced visualizations using different algorithms, such as a UMAP algorithm, t-distributed stochastic neighbor embedding (“t-SNE”) algorithm, principal components analysis (“PCA”) algorithm, or multidimensional scaling (“MDS”) algorithm;
The ability to color a visualization based on metadata such as clinical information (e.g., WHO grade) or demographic information (e.g., age, gender, ethnicity);
The ability to create and save patient cohorts by identifying (e.g., encircling) those patient cohorts through the visualization, for example, to allow for additional analysis of individual patient cohorts, comparisons of different patient cohorts, etc.;
The ability to observe characteristics (e.g., outcomes) of a given patient cohort through dynamic generation of visualizations (e.g., Kaplan-Meier survival plots to show probability of survival at different time intervals);
The ability to highlight specific patient cohorts within a visualization and remove specific patient cohorts from a visualization;
The ability to move a patient cohort en block or en masse out of the way within a visualization;
The ability to visually connect a patient or patient cohort to a second analysis by way of edges (e.g., wherein the interface is bifurcated or otherwise split between a UMAP model and chromosomes with genes that are mutated in the cohort); and
The ability to programmatically and visually connect with data (e.g., clinical information over time) for either the entire patient population or smaller patient cohorts.

With these abilities, the analysis platform allows for relationships between data say, the clinical information of a newly diagnosed patient and clinical information of other patients diagnosed as having the same disease—to be more readily presented in a coherent, comprehensible manner that allows those relationships to be used for improved development of personalized care plans.

In accordance with a more specific aspect, a method involves generating a landscape of RNA expression profiles from a plurality of patients (“the patients”) having a set of medical attributes (e.g., a medical diagnosis or related RNA expression profile attributes) that is common to the patients, for example as may relate to a medical condition such as cancer. An RNA expression profile is assessed for a target patient having the set of medical attributes. A plurality of nearest neighbor RNA expression profiles are identified from the landscape of RNA expression profiles, based on the target patient's RNA expression profile and by computing a distance corresponding to one or more centroid values pertaining to the patients. Data for a diagnosis for the target patient is generated and outputted based on known characteristics of the patients corresponding to the identified nearest neighbor RNA expression profiles. In these contexts, the diagnosis may relate to a diagnosis having greater specificity relative to a general condition exhibited by the patients (e.g., with a genus diagnosis of cancer and a more detailed diagnosis for more specific cancer-related aspects of the target patient). In addition, the target patient may or may not be from among the (plurality of) patients. Further, a “patient” in these contexts may involve a healthy person for which testing is performed.

As an example, a first set of attributes may correspond to margin tissue perceived as being clean relative to tumor tissue, and the target patient (whether or not from among the same patients) may be the subject of the steps of ascertaining, identifying and generating and outputting. In some instances, both clean and tumor tissue from the same patient are assessed for a landscape constructed from many “tumor” and “normal” samples.

In a more particular aspect, the landscape of RNA expression profiles is generated by arranging RNA sequence data from the plurality of patients in a lower dimensional representation. The plurality of nearest neighbor RNA expression profiles may be identified by obtaining a visualization that includes a plurality of visual indicia for the plurality of patients. A list is complied, which includes each existing patient of the plurality of patients that are established as nearest neighbors. Coordinates within the lower dimensional representation for the target patient are determined and used to establish one or more of the plurality of patients as nearest neighbors to the target patient in the lower dimensional representation. For instance, a user may identify clusters of patients having more detailed diagnoses that match (e.g., closely), and land the target patient onto one of the clusters that is a best match.

In a more particular aspects, a centroid value (e.g., in a visualization) for the target patient is identified by calculating a centroid of values corresponding to the patients in the list, and computing a distance from the centroid to the values corresponding to each existing patient of the plurality of patients. Such an approach may assess groupings of values for respective patients (e.g., in initial assessment steps, the computer-implemented method may be assessing a multitude (at least 100 or at least 1,000 in certain examples) of values for respective patients). The values corresponding to the plurality of patients are filtered based on the computed distance for each of the plurality of patients and a predetermined percentile in terms of the computed distance from the centroid, therein producing filtered values. The centroid (aka centroid value) is recalculated for the filtered values and the filtered values are further filtered based on a radius of the recalculated centroid, therein producing a remaining set of filtered values. A centroid is calculated as an approximate location for the target patient based on coordinates of the patients corresponding to the remaining set of filtered values. The centroid can then be used to assess the target patient's diagnosis.

In certain aspects, when the target patient is landed on data from existing patients, there may be variation or “jitter” due to the stochastic nature of a dimensionality-reduction algorithm used to provide the dimensionally-reduced data. The target patient may be recasted onto data from existing patients another time or multiple times (as one or more iterations), with each iteration used together to verify or more accurately provide identification of nearest neighbors, thereby mitigating or avoiding such variation as discussed above. For instance, based on one or more iterations of identifying from the landscape of RNA expression profiles and based at least in part on the target patient's RNA expression profile, the target patient may be landed within an n-sphere of specified radius, based on root-mean-square distance in n-space.

In some implementations, a value is computed for each of the plurality of patients, the value being selected from the group of: a frequency of a patient being established as a nearest neighbor, a count of N lower dimensional representations in which a patient was established as a nearest neighbor, and a combination thereof. The list of the plurality of patients is filtered by removing patients having characteristics selected from the group of: a frequency that is less than a threshold, a count that is less than a threshold, and a combination thereof. A centroid of values corresponding to the patients in the list is calculated and a distance from the centroid to each patient of the plurality of patients is computed. Based on the computed distance for each of the plurality of patients, a first set of patients that are above a predetermined percentile in terms of distance are identified and filtered so as to generate a first filtered set of patients. The centroid is recalculated for the first filtered set of patients, and a second set of patients that are located outside of a radius of the recalculated centroid are identified. The second set of patients is filtered from the first filtered set of patients so as to generate a second filtered set of patients. For each remaining patient that is not in the first or second sets of filtered patients, coordinates of the patients are identified and used to calculate a centroid, the coordinates of which may be ascertained as an appropriate location/value for the target patient.

In certain aspects, the landscape of RNA expression profiles are provided by arranging RNA sequence data from the plurality of patients in a lower dimensional representation includes transforming the RNA sequence data into a data set having a lower dimension than the RNA sequence data. A visualization may be obtained by plotting values corresponding to the lower dimensional representation. The plurality of nearest neighbor RNA expression profiles are identified by identifying clusters of the values (e.g., as may be plotted) and matching the target patient's RNA expression profile to one of the identified clusters.

Another aspect is directed to assessing the target patient's diagnosis by obtaining a visualization that includes visual indicia for the plurality of patients having the common medical diagnosis, and forming a plurality of clusters of the patients by clustering the visual indicia in the visualization. For each cluster, the patients are arranged in a lower dimensional representation by applying a graph layout algorithm to the visualization or to the RNA expression profiles for the patients in the cluster. Coordinates within the lower dimensional representation for the target patient are ascertained. Based on the coordinates, one or more of the plurality of patients are identified as nearest neighbors to the target patient in the lower dimensional representation, and the target patient is assigned to a cluster in which a majority of the patients identified as the plurality nearest neighbors reside.

In certain implementations, the plurality of nearest neighbor RNA expression profiles are identified by defining, in a visualization from a centroid corresponding to the plurality of patients, a line that is either representative of a radius of a circle or representative of a radius of a sphere. The plurality of patients within the circle or sphere are identified as members of a nearest neighbors cohort corresponding to the plurality of nearest neighbor RNA expression profiles.

In certain aspects, a centroid value is identified from a visualization that includes a plurality of visual indicia for the target patient as follows. A centroid of RNA expression profile values corresponding to the plurality of patients is calculated and a distance from the centroid to the values corresponding to each of the plurality of patients is determined. The values corresponding to the plurality of patients are filtered based on the computed distance for each of the plurality of patients and a predetermined percentile in terms of the computed distance from the centroid, therein producing filtered values. The centroid is recalculated for the filtered values and the filtered values are again filtered based on a radius of the recalculated centroid, therein producing a remaining set of filtered values. A centroid is then calculated as an approximate location for the target patient based on coordinates of the patients corresponding to the remaining set of filtered values.

The nearest neighbor RNA expression profiles may be identified in a variety of manners. In some implementations, these are identified as profiles of ones of the plurality of patients that are more similar to the target patient's RNA expression profile than RNA expression profiles of other ones of the plurality of patients.

In other implementations, the landscape of RNA expression profiles from the plurality of patients and the target patient's RNA expression profile are transformed into a lower dimensional representation, and the target patient's transformed RNA expression profile is matched to a subset (e.g., cluster) of the transformed RNA expression profiles from the plurality of patients. These steps of transforming and matching may be repeated multiple times, and the plurality of nearest neighbor RNA expression profiles can be identified based on the matched subsets from each of the repeated steps of matching. Such an approach may address issues such as noise or jitter that may affect the transformation, with the iterative calculations utilized to focus the nearest neighbor groups. In a specific application, a list is compiled to include each of the plurality of patients that is established as a nearest neighbor via at least one of the repeated steps of matching, and a frequency of each one of the plurality of patients as having been established as a nearest neighbor. A subset of the plurality of patients are identified as nearest neighbors based on the computed frequencies.

In certain specific applications, the detailed diagnosis includes a risk value characterizing the target patient's medical risk based on known characteristics including known medical risk for the plurality of patients of the nearest neighbor RNA expression profiles and medical records of the patient.

Another embodiment is directed to an apparatus having processing circuitry that generates a landscape of RNA expression profiles from a plurality of patients (“the patients”) having a set of medical attributes that is common to the patients. For a target patient having the set of medical attributes, the processing circuitry ascertains the target patient's RNA expression profile and identifies a plurality of nearest neighbor RNA expression profiles from the landscape of RNA expression profiles, based on the target patient's RNA expression profile. This may include computing a distance corresponding to one or more centroid values pertaining to RNA expression profile values for the patients. Data for a diagnosis for the target patient is generated and outputted based on known characteristics of the patients corresponding to the identified nearest neighbor RNA expression profiles

In a more specific application, the processing circuitry generates the landscape of RNA expression profiles by arranging RNA sequence data from the plurality of patients in a lower dimensional representation, and identifies the plurality of nearest neighbor RNA expression profiles as follows. A visualization that includes a plurality of visual indicia for the plurality of patients may be obtained and utilized in this regard. A list that includes each existing patient of the plurality of patients that are established as nearest neighbors is compiled. Coordinates within the lower dimensional representation are identified for the target patient, and one or more of the plurality of patients is established as nearest neighbors to the target patient in the lower dimensional representation, based on the coordinates.

The processing circuitry may identify a centroid value for the target patient by calculating a centroid of values corresponding to the patients in the list, and computing a distance from the centroid to the values corresponding to each existing patient of the plurality of patients. The values corresponding to the plurality of patients are filtered based on the computed distance for each of the plurality of patients and a predetermined percentile in terms of the computed distance from the centroid, therein producing filtered values. The centroid for the filtered values is then calculated, and the filtered values are filtered further based on a radius of the recalculated centroid, therein producing a remaining set of filtered values. A centroid is calculated as an approximate location for the target patient, based on coordinates of the patients corresponding to the remaining set of filtered values.

Further, the processing circuitry may compute, for each patient in the list, a value selected from the group of: a frequency of a patient being established as a nearest neighbor, a count of N lower dimensional representations in which a patient was established as a nearest neighbor, and a combination thereof. The processing circuitry then filters the list by removing patients having characteristics selected from the group of: a frequency that is less than a threshold, a count that is less than a threshold, and a combination thereof. A centroid of values corresponding to the patients in the list is calculated and a distance from the centroid to each patient of the plurality of patients is determined. Based on the computed distance for each of the plurality of patients, a first set of patients that are above a predetermined percentile in terms of distance is identified and filtered so as to generate a first filtered set of patients. The centroid is recalculated for the first filtered set of patients and a second set of patients that are located outside of a radius of the recalculated centroid are identified and filtered so as to generate a second filtered set of patients. For each remaining patient that is not in the first or second sets of filtered patients, coordinates of the patients are identified, a centroid is calculated based on the coordinates identified for the remaining patients, and coordinates of the centroid are established as an appropriate location for the target patient. Such approaches may utilize visualizations as characterized herein.

In certain aspects, the processing circuitry generates the landscape of RNA expression profiles by arranging RNA sequence data from the plurality of patients in a lower dimensional representation, which includes transforming the RNA sequence data into a data set having a lower dimension than the RNA sequence data. A visualization may be obtained by plotting values corresponding to the lower dimensional representation. Clusters of the plotted values are identified, and the target patient's RNA expression profile is matched to one of the identified clusters (as nearest neighbor RNA expression profiles).

The processing circuitry may assess diagnoses by obtaining a visualization that includes visual indicia for the plurality of patients having a common medical diagnosis or other medical attributes. A plurality of clusters of the patients is formed, for instance by clustering visual indicia in a visualization. For each cluster, the patients are arranged in a lower dimensional representation by applying a graph layout algorithm to the visualization or to the RNA expression profiles for the patients in the cluster. Coordinates within the lower dimensional representation for the target patient are ascertained and one or more of the plurality of patients are identified as nearest neighbors to the target patient in the lower dimensional representation, based on the coordinates. The target patient is assigned to a cluster in which a majority of the patients identified as the plurality nearest neighbors reside.

The processing circuitry may identify the plurality of nearest neighbor RNA expression profiles by defining, in a visualization from a centroid corresponding to the plurality of patients, a line that is either representative of a radius of a circle or representative of a radius of a sphere. The plurality of patients within the circle or sphere are identified as members of a nearest neighbors cohort corresponding to the plurality of nearest neighbor RNA expression profiles.

In another specific aspect, the processing circuitry identifies the plurality of nearest neighbor RNA expression profiles based on the target patient's RNA expression profile, by identifying RNA expression profiles of ones of the plurality of patients that are more similar to the target patient's RNA expression profile than RNA expression profiles of other ones of the plurality of patients.

Furthermore, aspects of the present disclosure are directed to systems and methods that implement trained AI processing involving the processing of patient data and related matching/landing. For instance, application of trained AI processing (e.g., one or more trained machine learning models) may be adapted to evaluate data pertaining to clustering patient data relative to diagnoses, and for iteratively assessing a target patient's diagnosis relative to such clusters. In some examples, one or more components are configured to manage the application of one or more AI models to enhance processing described in the present disclosure. Trained AI processing is applicable to aid determinative or predictive processing including specific processing operations described with respect to providing a detailed diagnosis and/or to predict patient risk for certain medical conditions. An exemplary component for implementation trained AI processing may manage AI modeling including the creation, training, application, and updating of AI modeling. Trained AI processing may be adapted to execute specific determinations described herein including those for analyzing specific data and data sources of a software data platform (e.g., a medical history software platform) and/or generating insights for data augmentation. For instance, an AI model may be specifically trained and adapted for execution of processing operations pertaining to the generation of a landscape, groupings and landing of a target patient within such a landscape. In one example, trained AI processing comprises a hybrid AI model (e.g., hybrid machine learning model) that is adapted and trained to execute a plurality of processing operations described in the present disclosure. In alternative examples, trained AI processing comprises a collective application of a plurality of trained AI models that are separately trained and managed to execute processing described herein. In examples where a plurality of independently trained and managed AI models is implemented, downstream processing efficiency may be improved by an ordered application of trained AI models where processing results from earlier applied AI models can be propagated to subsequently applied AI models. For example, a trained AI model may evaluate transformed (lower dimensional) data and derive data correlations to improve processing and efficiency, which may then be utilized to suggest a re-prioritization of opportunities (and/or reallocation of resources as may be appropriate) to improve efficiency and quality of services provided.

Non-limiting examples of supervised learning that may be applied comprise but are not limited to: nearest neighbor processing; naive bayes classification processing; decision trees; linear regression; support vector machines (SVM) neural networks (e.g., convolutional neural network (CNN) or recurrent neural network (RNN)); and transformers, among other examples. Non-limiting examples of unsupervised learning that may be applied comprise but are not limited to: application of clustering processing including k-means for clustering problems, hierarchical clustering, mixture modeling, etc.; application of association rule learning; application of latent variable modeling; anomaly detection; and neural network processing, among other examples. Non-limiting examples of semi-supervised learning that may be applied comprise but are not limited to: assumption determination processing; generative modeling; low-density separation processing and graph-based method processing, among other examples. Non-limiting examples of reinforcement learning that may be applied comprise but are not limited to: value-based processing; policy-based processing; and model-based processing, among other examples. Furthermore, a component for implementation of trained AI processing may be configured to apply a ranker to generate relevance scoring to assist with any processing determinations with respect to any relevance analysis, such as that described herein. Scoring for relevance (or importance) ranking may be based on individual relevance scoring metrics described herein or an aggregation of said scoring metrics. In some examples where multiple relevance scoring metrics are utilized, a weighting may be applied that prioritizes one relevance scoring metric over another depending on the signal data collected and the specific determination being generated.

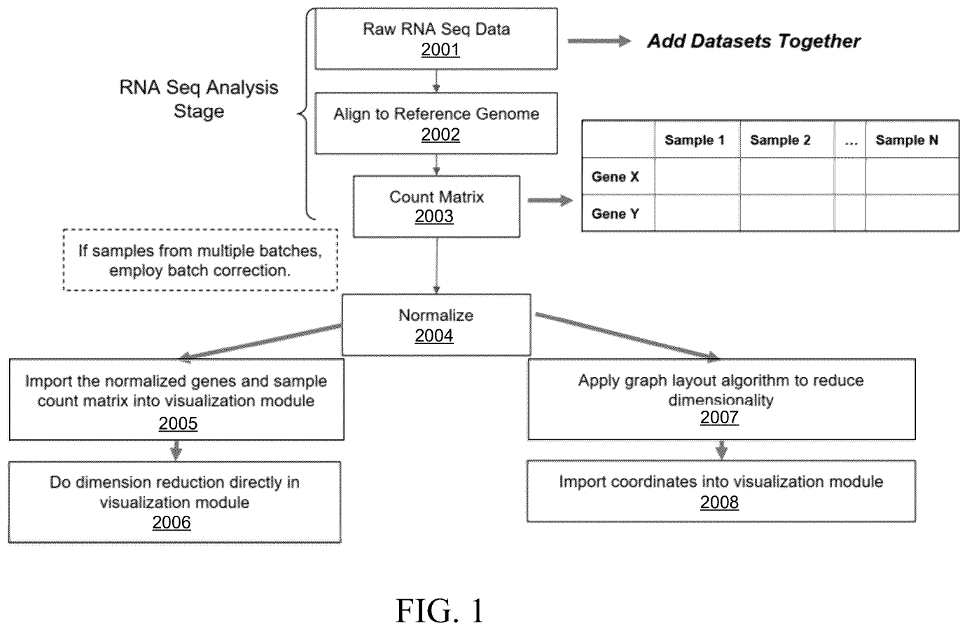

Turning now to the Figures, FIG. 1 includes a flow diagram of a workflow 2000 for generating a visualization based on RNA seq data. Initially, the analysis platform can obtain raw RNA seq data from one or more sources (step 2001). Generally, the analysis platform acquires raw RNA seq data from multiple sources as mentioned above, and therefore the analysis platform may concatenate multiple datasets in a single data structure to create a superset. In some embodiments, each RNA seq data is in the FASTQ format, allowing the analysis platform to more consistently and accurately create the superset. FASTQ format is a text-based format for storing a biological sequence—usually a nucleotide sequence—and its corresponding quality scores. Both the sequence letter and quality score can be encoded with a single ASCII character for brevity.

Thereafter, the analysis platform can align the RNA seq data with a reference genome (step 2002)—Hg38 for human or another species—using Spliced Transcripts Alignment to a Reference (“STAR”), for example. STAR is a fast RNA-seq read mapper, with support for splice junction and fusion read detection. At a high level, STAR aligns reads by finding the Maximal Mappable Prefix (“MMP”) hits between reads (or read pairs) and the reference genome, using a Suffix Array index. Different parts of a read can be mapped to different genomic positions, corresponding to splicing or RNA fusions. The genome index includes known splice junctions from annotated gene models, allowing for sensitive detection of spliced reads.

The analysis platform can then generate a count matrix based on an analysis of the RNA seq data as aligned with the reference genome (step 2003). The count matrix may be representative of a data structure (e.g., a table) that specifies, for each gene of interest, a count for each sample in the RNA seq data. In embodiments where the RNA seq data includes samples from multiple batches, the analysis platform can perform patch correction as discussed above. Specifically, the analysis platform may utilize ComBat-seq, which is a negative binomial regression model that retains the integer nature of count data in RNA seq data, making the batch-adjusted RNA seq data compatible with common differential expression software packages that require integer counts.

The analysis platform may also perform a normalization operation on the RNA seq data (step 2004). For example, the analysis platform may take the log2 of the Transcript Count Per Million (“TPM”).

In some embodiments, the analysis platform imports the normalized genes and sample count matrix into the visualization module (step 2005) and then performs dimensionality reduction directly in the visualization module (step 2006). In other embodiments, the analysis platform applies a graph layout algorithm to the normalized genes and sample count matrix to reduce dimensionality (step 2007) and then imports the coordinates (e.g., x-, y-, and z-coordinates) into the visualization module (step 2008). Note that the analysis platform could important the coordinates into another computer program instead of, or in addition to, the visualization module. This other computer program could be executing on the same computing device as the analysis platform or another computing device.

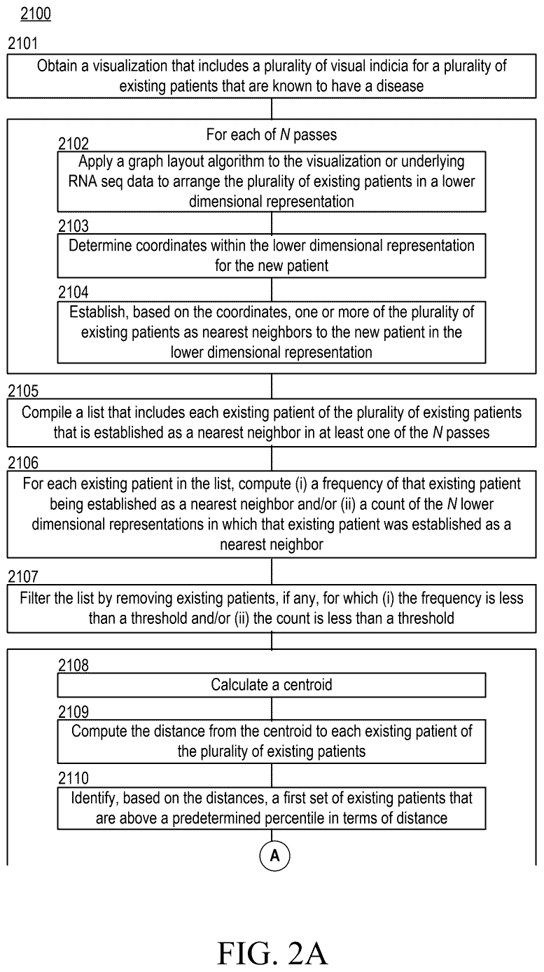

FIGS. 2A and 2B show a flow diagram of a process 2100 for overlaying a patient newly diagnosed with a disease (e.g., meningioma) on a visualization, so as to visually illustrate the relationship with a plurality of existing patients known to have the disease. Initially, the analysis platform can obtain the visualization that includes a plurality of visual indicia for the plurality of existing patients that are known to have the disease (step 2101). One example of such a visualization is shown in FIG. 1 of the '717 Provisional. Note, however, that the visualization shown is based on RNA seq data for multiple types of brain tumors. In the visualization, the location of each visual indicium of the plurality of visual indicia may be based on an analysis of RNA seq data associated with a corresponding existing patient of the plurality of existing patients. More generally, the location of each visual indicium may be based on an analysis of health-related information (e.g., RNA seq data plus other information, such as physiological information, treatment information, or contextual information) associated with a corresponding one of the plurality of existing patients.

For each pass of N passes, the analysis platform can perform a series of actions. First, the analysis platform can apply a graph layout algorithm to the visualization or underlying RNA seq data to arrange the plurality of existing patients in a lower dimensional representation (step 2102). For example, the analysis platform may implement a UMAP algorithm that, upon being applied to the visualization or underlying RNA seq data, constructs a higher dimensional representation and then optimizes the lower dimensional representation to be as structurally similar to the higher dimensional representation as possible. In such embodiments, the analysis platform may employ the UMAP algorithm with a different random seed for each pass of the N passes. Meanwhile, structural similarity could be based on cross entropy as measured between the lower and higher dimensional representations produced by the UMAP algorithm.

Second, the analysis platform can determine coordinates within the lower dimensional representation for the new patient (step 2103). The analysis platform can accomplish this through an analysis of RNA seq data associated with the new patient, in order to determine where best to locate the new patient in the visualization. For example, the analysis platform may put the RNA seq data associated with the new patient through the same UMAP model used to create the visualization, as discussed above with reference to FIG. 1 in the '717 Provisional, in order to establish the coordinates.

Third, the analysis platform can establish, based on the coordinates, one or more of the plurality of existing patients as nearest neighbors to the new patient in the lower dimensional representation (step 2104).

Generally, it is helpful to remove “noisy” nearest neighbors in order to lessen the likelihood that these nearest neighbors affect insights drawn from the placement of the new patient in the visualization. Accordingly, the analysis platform may compile a list that includes each existing patient of the plurality of existing patients that is established as a nearest neighbor in at least one of the N passes (step 2105). For each existing patient in the list, the analysis platform can compute (i) a frequency of that existing patient being established as a nearest neighbor and/or (ii) a count of the N lower dimensional representations in which that existing patient was established as a nearest neighbor (step 2106). The analysis platform can then filter the list accordingly. Specifically, the analysis platform can filter the list by removing existing patients, if any, for which (i) the frequency is less than a threshold (e.g., at least 40, 60, or 80 percent) and/or (ii) the count is less than a threshold (e.g., if N is 10, the count might be 3 or 5; if N is 100, the count might be 20, 30, or 50; etc.) (step 2107). Accordingly, the list could be filtered based on the frequency with which existing patients are established as nearest neighbors or the total count of times that existing patients are established as nearest neighbors. In some embodiments, filtering based on frequency may be preferred because it is easier to scale up as more patients are added to the visualization, and therefore continued performance of the process 2100 may be less computationally burdensome.

Of the existing patients remaining in the filtered list, the analysis platform can remove those existing patients that are determined to be outliers. Again, removing outliers may require performance of a series of actions. First, for each lower dimensional representation of the N lower dimensional representations, the analysis platform can calculate a centroid (step 2108) and then compute the distance from the centroid to each existing patient of the plurality of existing patients (step 2109). The analysis platform can then identify, based on the distances, a first set of existing patients that are above a predetermined percentile in terms of distance from the centroid (step 2110). For example, the analysis platform may identify those existing patients below the 85th, 90th, or 95th percentile. The analysis platform can filter the first set of existing patients from the plurality of existing patients so as to generate a first filtered set of patients (step 2111), and then the analysis platform can recalculate the centroid for the first filtered set of patients (step 2112). Such an approach ensures that the recalculated centroid is no longer influenced by those existing patients that are beneath the predetermined percentile. Moreover, the analysis platform may identify a second set of existing patients that are located outside of a radius of the recalculated centroid (step 2113), and then the analysis platform may filter the second set of existing patients from the first filtered set of patients so as to generate a second filtered set of patients (step 2114). At a high level, the analysis platform can “chop” (e.g., remove or ignore) existing patients that fall outside of the radius, and therefore are representative of outliers. Several different approaches could be employed by the analysis platform to compute, establish, or otherwise determine the radius. In some embodiments, the analysis platform normalizes the visualization so that the average distance between every visual indicium is one and then implements a radius of fixed value (e.g., 5, 10, 15). In other embodiments, the radius is adaptive, for example, based on the number of existing patients, the total spread of the existing patients, etc. An adaptive radius is generally not well suited for clusters with few members or clusters that are close to, or intermingled with, other clusters because it is difficult for the analysis platform to automatically determine the appropriate cutoff. Instead, an adaptive radius is generally a better option where the existing patients in the visualization tend to be separate “islands” with good density (e.g., at least 20, 50, or 100 existing users collocated together in close proximity).

For each remaining existing patient, the analysis platform can identify coordinates of that existing patient in the visualization (step 2115). Then, the analysis platform can calculate a centroid based on the coordinates identified for the remaining existing patients (step 2116). The analysis platform can establish coordinates of the centroid as an appropriate location for the new patient in the visualization (step 2117). Accordingly, the analysis platform could associate the coordinates of the centroid with the new patient in a data structure that is representative of a digital profile maintained for the new patient, and the analysis platform could post, to the visualization, a visual indicium that is representative of the new patient at the coordinates of the centroid. The visual indicium that is representative of the new patient could be visually distinguishable from the visual indicia that are representative of the remaining existing patients. For example, the visual indicium that is representative of the new patient could be rendered in a different color, with greater saturation, in a different shape, etc. As mentioned above, in the visualization, each remaining existing patient can be represented by a separate visual indicium. In some embodiments, the appearances of these visual indicia are based on the frequency or count of the N lower dimensional representations in which that remaining existing patient was established as a nearest neighbor. Such an approach may allow a user to more readily understand which remaining existing patients are determined to be the closest matches to the new patient.

Additional information on the process for overlaying a new patient onto the visualization that is serves as a reference landscape can be found in Appendix B of the '717 Provisional.

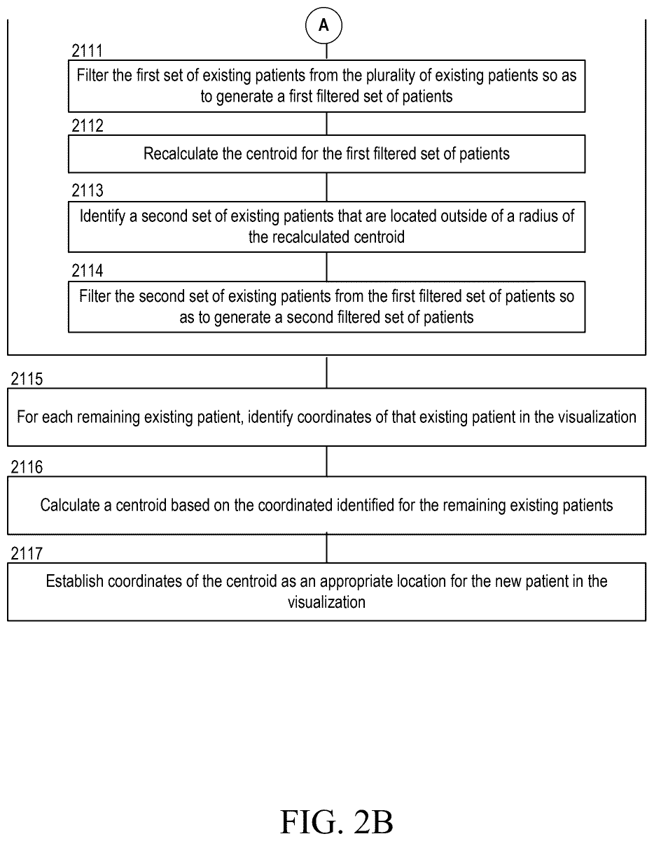

FIG. 3 shows a flow diagram of a process 2200 for improving classification of a patient newly diagnosed with a disease (e.g., meningioma). After performing the process 2100 of FIGS. 2A and 2B, the analysis platform may be tasked with identifying the cluster with which the new patient is most closely associated. To accomplish this, the analysis platform can identify the cluster to which the majority of the existing patients identified as nearest neighbors belong to and then assigning the new patient to this identified cluster. This results in fewer incorrect predictions on cluster boundaries, which is a known issue for nearest neighbor-based approaches.

Accordingly, the analysis platform can obtain a visualization that includes visual indicia for existing patients that are known to have a disease (step 2201) and then form a plurality of clusters by clustering the visual indicia in the visualization (step 2202). To form the plurality of clusters, the analysis platform may apply, to the visualization or underlying RNA seq data, a clustering algorithm that defines boundaries such that similar existing patients (e.g., based on RNA seq data, disease classification, disease outcome) are grouped together. For each pass of N passes, the analysis platform can apply a graph layout algorithm to the visualization to arrange the existing patients in a lower dimensional representation (step 2203), determine coordinates within the lower dimensional representation for the new patient (step 2204), and identify, based on the coordinates, one or more of the existing patients as nearest neighbors to the new patient in the lower dimensional representation (step 2205). In response to determining that a majority of the existing patients identified as nearest neighbors reside in a given cluster of the plurality of clusters (step 2206), the analysis platform can assign the new patient to the given cluster (step 2207). For example, the analysis platform may populate, into a data structure associated with the new patient, information that is associated with, or representative of, the cluster. This information could be a severity classification (e.g., WHO grade), a treatment classification, a predicted outcome, etc.

In a specific implementation, aspects of FIG. 3 are implemented as follows with a computer-implemented method. A landscape of RNA expression profiles is generated from a plurality of patients a having a set of one or more medical attributes that is common to the patients, for example as part of step 2201 (and e.g., 2202). For a target patient having the set of medical attributes, an RNA expression profile of the target patient is ascertained and a plurality of nearest neighbor RNA expression profiles are identified from the landscape of RNA expression profiles. This may be carried out based on the target patient's RNA expression profile and at least one computed distance corresponding to at least one centroid value for the RNA expression profiles, for example in connection with steps 2203-2205. Data for a diagnosis or other health-specific recommendation can be generated and output for the target patient, based on known characteristics of the patients corresponding to the identified plurality of nearest neighbor RNA expression profiles. For instance, steps 2206 and 2207 may be implemented to identify a cluster and to assign the target patient to a particular cluster, with the diagnosis or health-specific recommendation being generated in accordance with such information for the identified and assigned cluster and output in step 2208.

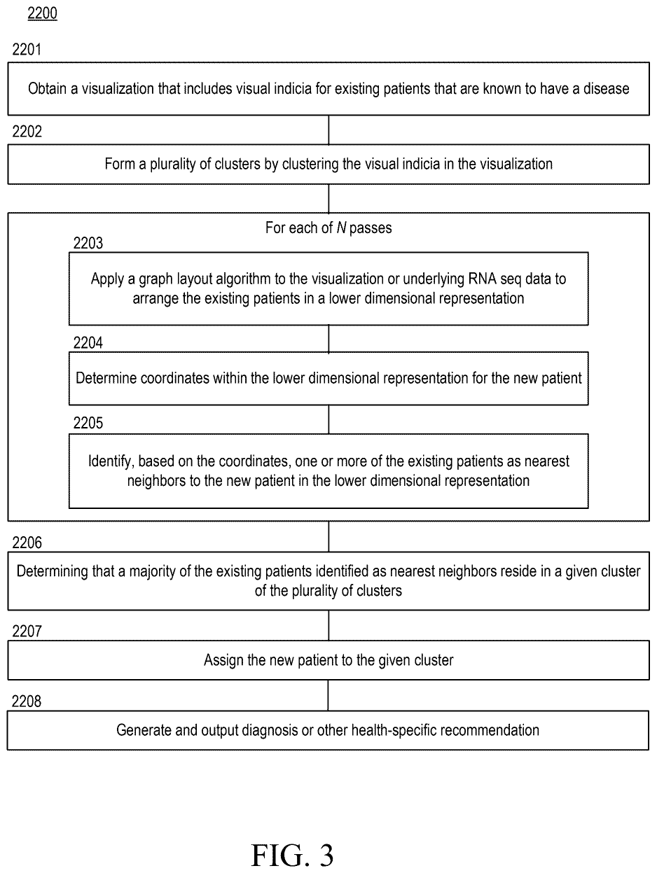

FIG. 4 shows a flow diagram of a process 2300 for identifying a cohort of nearest neighbors and producing an appropriate output. After performing the process 2100 of FIGS. 2A and 2B, the analysis platform may be tasked with gaining insights into the cohort of nearest neighbors. To accomplish this, the analysis platform may define, in the visualization from the centroid, a line that is either representative of a radius of a circle (i.e., in the event that the visualization is in two dimensions) or a radius of a sphere (i.e., in the event that the visualization is in three dimensions) (step 2301). The analysis platform can define all existing patients within the circle or sphere as members of a nearest neighbors cohort (step 2302). Upon identifying the nearest neighbors cohort, additional actions could be taken. For example, the analysis platform could recalculate metrics (e.g., survival likelihood) or plots (e.g., survival plot) such that only members of the nearest neighbors cohort are considered. This could be done dynamically in response to establishing the centroid as discussed above with respect to FIGS. 2A and 2B. Moreover, the metrics or plots may be interactive such that as new patients are added—and the nearest neighbors cohort is updated—immediate changes and longer term trends are identifiable.

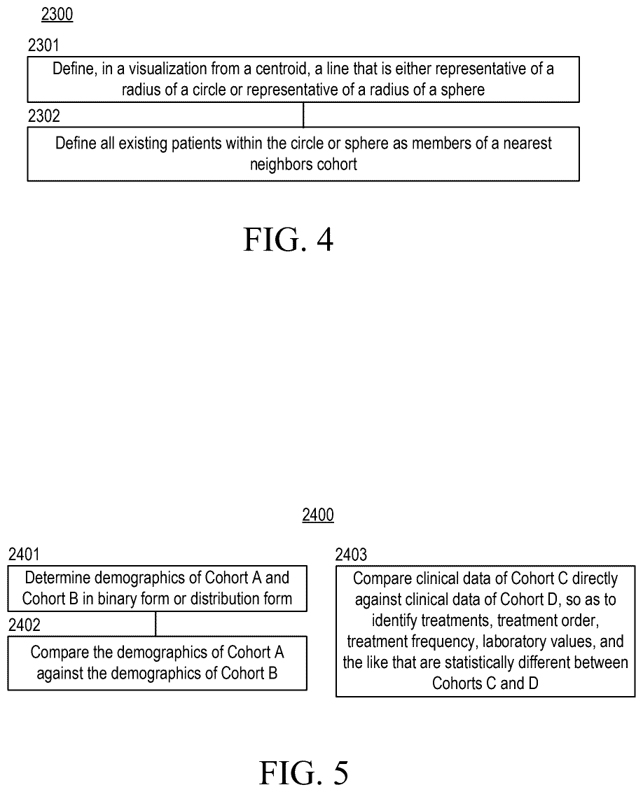

FIG. 5 shows a flow diagram of a process 2400 for comparing two selected cohorts of patients, for example, through subtraction of an average value versus another average value or differential gene expression. The nature of the comparison may depend on the nature of the underlying data.

Assume, for example, that the analysis platform is interested in comparing the demographics (e.g., gender, age distribution, race or ethnicity) of two cohorts, namely, Cohort A and Cohort B. In such a scenario, the analysis platform can determine the demographics of Cohorts A and B are either binary (e.g., percentage with or without a given characteristic) or distribution (e.g., as a histogram) (step 2401) and then directly compare the demographics of Cohort A against the demographics of Cohort B (step 2402). Such an approach is generally dependent on demographic data either accompanying the RNA seq data of the patients in Cohorts A and B or otherwise being obtained by the analysis platform (e.g., derived from corresponding electronic health records).

As another example, assume that the analysis platform is interested in comparing the clinical data of two cohorts, namely, Cohort C and Cohort D. In such a scenario, the analysis platform may directly compare the clinical data of Cohort C to the clinical data of Cohort D, so as to identify treatments, treatment order, treatment frequency, laboratory values, and the like that are statistically different between Cohorts C and D (step 2403). For example, the analysis platform may automatically compare static clinical attributes—like age, sex, and presence or absence of features—by computing an appropriate statistical test and then sorting comparisons by significance. Additionally or alternatively, the analysis platform may automatically compare events—like prescribing of medication, performing of testing procedures, and performing of treatment procedures—based on the presence or absence of such events, or the frequency of such events, as well as event-based values that are more readily trackable and comparable. Examples of event-based values include the values of diagnostic tests, medication dosage, radiation dosage, and the like.

In addition to these computational comparisons, the analysis platform could also permit visual comparisons of different cohorts. For example, the analysis platform may be able to visualize clinical data associated with different cohorts in the form of a bar graph, line graph, or another form of diagram. These diagrams may be helpful in allowing different cohorts—which could include tens, hundreds, or thousands of patients—in a rapid manner.

As another example, assume that the analysis platform is interested in comparing the molecular data of two cohorts, namely, Cohort E and Cohort F. In such a scenario, the analysis platform may use a statistical tool that produces, for Cohorts E and F, a data structure (e.g., an S-table with P-values) populated with GO term analysis of differential gene expression. Moreover, the analysis platform may identify the differential copy numbers between Cohorts E and F and present the same, for example, in the form of a Manhattan plot. Manhattan plots could be dynamically generated in response to receiving input that is indicative of a selection of one or more cohorts. For example, upon receiving input that is indicative of a selection of a cohort, the analysis platform may calculate a copy-number Manhattan plot from the corresponding RNA seq data as a thumbnail plot that is interactable and movable. Similarly, the analysis platform may identify the differential mutations between Cohorts E and F and present the same, for example, as a list of mutations and corresponding P-values.

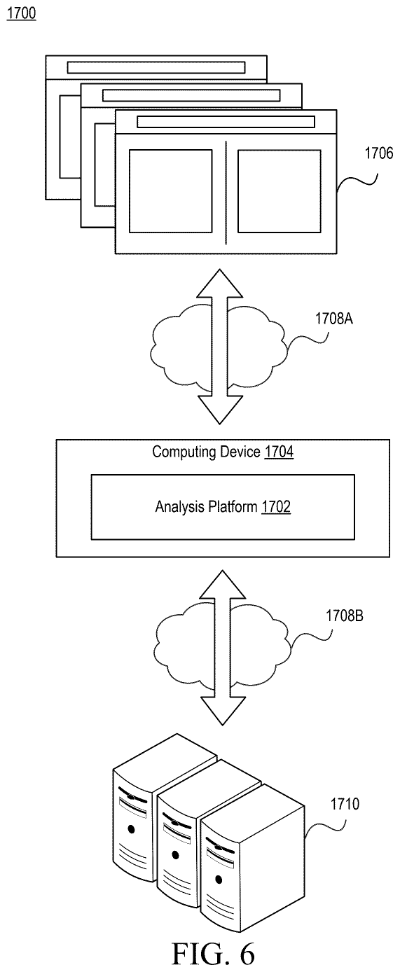

FIG. 6 shows a network environment 1700 that includes an analysis platform 1702 that is executed by a computing device 1704. An individual (also referred to as a “user”) can interact with the analysis platform 1702 via interfaces 1706. For example, a user may be able to access an interface through which visualizations of RNA seq data can be viewed and insights—derived by the analysis platform 1702 or user—can be documented. As another example, a user may be able to access an interface through sources of RNA seq data can be identified and processing of the RNA seq data can be overseen.

As shown in FIG. 6, the analysis platform 1702 can reside in a network environment 1700. Thus, the computing device 1704 on which the analysis platform 1702 resides can be connected to one or more networks 1708A-B. Depending on its nature, the computing device 1704 could be connected to a personal area network (“PAN”), local area network (“LAN”), wide area network (“WAN”), metropolitan area network (“MAN”), or cellular network. For example, if the computing device 1704 is a computer server, then the computing device 1704 may be accessible to users via respective mobile phones that are connected to the Internet via LANs. RNA seq data to be examined by the analysis platform 1702 may be acquired from sources external to the computing device 1704, such as network-accessible databases maintained by research organizations, academic institutions, healthcare systems, etc. Alternatively, the computing device 1704 could be associated with, and accessible to, a user—in which case the computing device 1704 may be connected to a server system 1710 that is responsible for supporting the analysis platform 1702. In such embodiments, the computing device 1704 could be a mobile phone, tablet computer, or wearable computing device (e.g., a fitness tracker or watch), for example.

Additionally or alternatively, the computing device 1704 may be connected to one or more other computing devices over a short-range wireless connectivity technology, such as Bluetooth®, Near Field Communication (“NFC”), Wi-Fi® Direct (also referred to as “Wi-Fi P2P”), and the like. As an example, the analysis platform 1702 could be embodied as a desktop application that is executed by a laptop computer. In such embodiments, the laptop computer may be communicative connected—via a wireless communication channel—to one or more sources from which to acquire RNA seq data. For example, the laptop computer may acquire RNA seq data from the server system 210, or the laptop computer may acquire RNA seq data from one or more network-accessible databases as mentioned above.

The interfaces 1706 may be accessible via a web browser, desktop application, mobile application, or another form of computer program. For example, if the user is a patient that has been recently diagnosed as having a brain tumor, the user may be able to access interfaces through which analysis of her own RNA seq data can be reviewed via a mobile application executing on a mobile phone. As another example, if the user is a healthcare professional, the user may be able to access interfaces through analysis of the RNA seq data of one or more patients can be reviewed via a desktop application executing on a tablet computer, laptop computer, or mobile workstation.

Generally, the analysis platform 1702 is executed—at least partially—by a cloud computing service operated by, for example, Amazon Web Services®, Google Cloud Platform™, or Microsoft Azure®. Thus, the computing device 1704 may be representative of a computer server that is part of a server system 1710. Often, the server system 1710 is comprised of multiple computer servers. These computer servers can include different types of data (e.g., RNA seq data for patients and additional information, such as name, demographic information, disease classification, disease treatment, disease outcome, etc.), algorithms for processing incoming data, algorithms for producing visualizations based on the processed data, and other assets. Those skilled in the art will recognize that these data could also be distributed among the server system 1710 and one or more computing devices. For example, some data that is input by, or related to, users may be stored on, and processed by, their own computing devices for security or privacy purposes.

Components of the analysis platform 1702 could also be hosted locally. That is, part of the analysis platform 1702 may reside on the computing device used to access one of the interfaces 1706. For example, the analysis platform 1702 may be embodied as a mobile application executing on a mobile phone as mentioned above. Note, however, that the mobile application may be communicatively connected to the server system 1710 on which other components of the analysis platform 1702 are hosted.

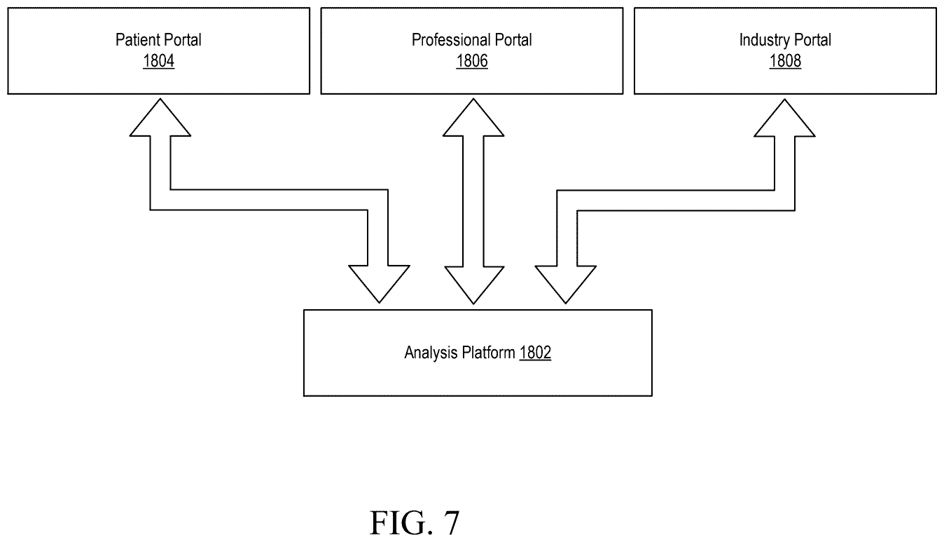

FIG. 7 shows an analysis platform 1802 may be accessible to different types of users via corresponding portals. Here, for example, the analysis platform 1802 is accessible via a patient portal 1804, healthcare professional portal 214 (or simply “professional portal”), and industry portal 216. At a high level, each portal may be representative of a set of interfaces that are designed to allow, promote, or other facilitate engagement with the corresponding set of users.

For example, the patient portal 1804 (also called the “patient platform” or “patient module”) may include interfaces through which patients can review their own information and personalized care plans, examine other deidentified patients identified as nearest neighbors by the analysis platform 1802, and view analyses (e.g., predictions) produced by the analysis platform 1802. In some instances, some information available through the patient portal 1804, like the names of other patients and dates of treatment, may be deidentified by obfuscating obscuring, encrypting, and/or otherwise making unavailable for privacy purposes.

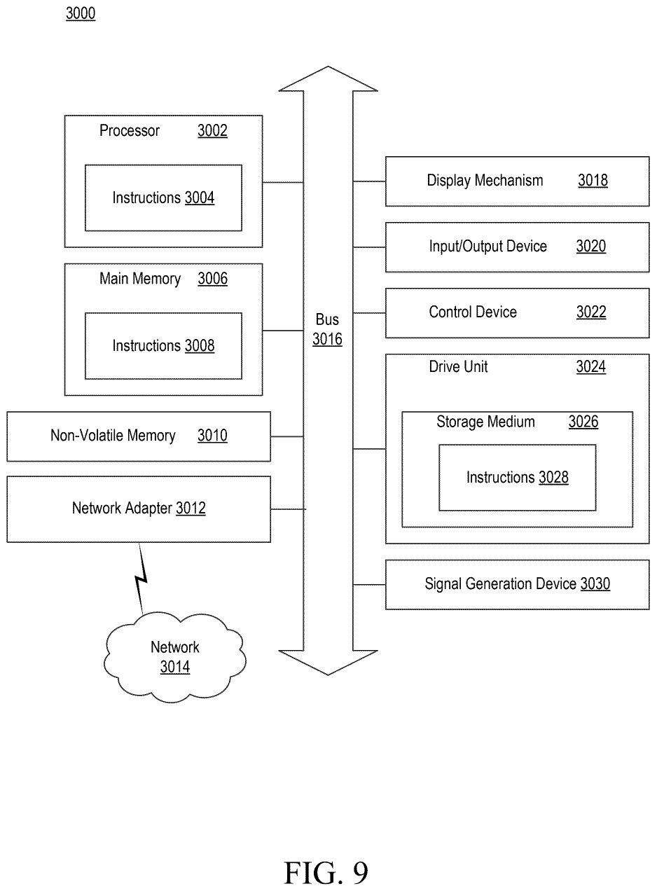

The professional portal 1806 may be designed for healthcare professionals, from specialists like oncologists to generalists like nurses and nurse practitioners, to review information associated with patients. Consider, for example, a scenario where a healthcare professional is tasked with developing a personalized care plan for a patient that was diagnosed as having cancer. To develop the personalized care plan, the healthcare professional may review the interfaces shown in FIG. 9 and in FIGS. 31-34 in the '717 Provisional, for example, and an industry professional (e.g., a developer or researcher) may review the interfaces shown to develop an understanding of how treatments affect outcomes across a population of patients. Through the interfaces, the healthcare professional may be able to establish nearest neighbors for the patient, review the treatments and outcomes of those nearest neighbors, and specify characteristics of the personalized care plan. These characteristics may be derived, by either the healthcare professional or the analysis platform 1802, through analysis of those nearest neighbors and corresponding information (e.g., the treatments and outcomes).

The industry portal 1808 may be designed for industry professionals, such as developers of pharmaceuticals and researchers. Through the interfaces accessible via the industry portal 1808, an industry professional may be able to monitor impact of a pharmaceutical during a trial by observing how outcomes of patients prescribed the pharmaceutical, as identified by the analysis platform 1802, are affected. As another example, an industry professional may be able to establish trends (e.g., in terms of outcomes) through analysis of a population of patients that are viewable via an interface. These trends may be helpful in developing pharmaceuticals (e.g., by identifying the specific pharmaceuticals or types of pharmaceuticals that tend to correlate with desirable outcomes, or by identifying patients—and characteristics thereof—that have undesirable outcomes or poor reactions to a specific pharmaceutical or type of pharmaceutical).

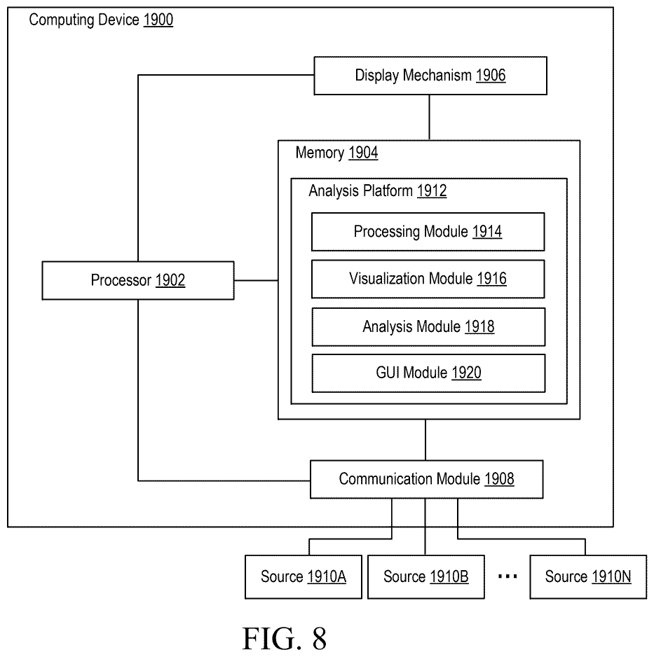

FIG. 8 shows an example of a computing device 1900 that is able to implement an analysis platform 1912 designed to produce visualizations based on RNA seq data. These visualizations can be helpful in gaining insights into patients that have been diagnosed as having brain tumors, as well providing feedback on the appropriate steps to take with respect to a patient that has been newly diagnosed as having a brain tumor as discussed below. As shown in FIG. 8, the computing device 1900 can include a processor 1902, memory 1904, display mechanism 1906, and communication module 1908. Each of these components is discussed in greater detail below.

Those skilled in the art will recognize that different combinations of these components may be present depending on the nature of the computing device 1900. For example, if the computing device 1900 is a computer server that is part of a server system (e.g., server system 1710 of FIG. 6), then the computing device 1900 may not include the display mechanism 1906. Conversely, if the computing device 1900 is a mobile phone or laptop computer, then the computing device 1900 can include the display mechanism 1906.

The processor 1902 can generic characteristics similar to general-purpose processors, or the processor 1902 may be an application-specific integrated circuit (“ASIC”) that provides control functions to the computing device 1900. The processor 1902 can be coupled to all components of the computing device 1900, either directly or indirectly, for communication purposes.

The memory 1904 can be comprised of any suitable type of storage medium, such as static random-access memory (“SRAM”), dynamic random-access memory (“DRAM”), electrically erasable programmable read-only memory (“EEPROM”), flash memory, or registers. In addition to storing instructions that can be executed by the processor 1902, the memory 1904 can also store data generated by the processor 1902 (e.g., when executing the modules of the analysis platform 1912). Note that the memory 1904 is merely an abstract representation of a storage environment. The memory 1904 could be comprised of actual integrated circuits (also called “chips”).

The display mechanism 1906 can be any mechanism that is operable to visually convey information to a user. For example, the display mechanism 1906 can be a panel that includes light-emitting diodes (“LEDs”), organic LEDs, liquid crystal elements, or electrophoretic elements, and/or may include a touchscreen. As discussed above, visualizations can be produced by the analysis platform 1912 (e.g., through execution of its modules), and these visualizations can be posted to the display mechanism 1906 for review by a user of the computing device 1900. As mentioned above, the user could be a patient whose RNA seq data is being examined, or the user could be a healthcare professional that is interested in reviewing RNA seq data associated with one or more patients.

The communication module 1908 may be responsible for managing communications external to the computing device 1900. The communication module 1908 can be wireless communication circuitry that is able to establish wireless communication channels with other computing devices. Examples of wireless communication circuitry include 2.4 gigahertz (“GHz”) and 5.8 GHz chipsets compatible with Institute of Electrical and Electronics Engineers (“IEEE”) 802.11—also referred to as “Wi-Fi chipsets.” Alternatively, the communication module 1908 may be representative of a chipset configured for Bluetooth, NFC, and the like. Some computing devices—like mobile phones, tablet computers, and the like—are able to wirelessly communicate via separate channels, while other computing devices—like servers and mobile workstations—tend to wirelessly communicate via a single channel. Accordingly, the communication module 1908 may be one of multiple communication modules implemented in the computing device 1900, or the communication module 1908 may be the only communication module implemented in the computing device 1900.

The nature, number, and type of communication channels established by the computing device 1900—and more specifically, the communication module 1908—can depend on (i) the sources from which data is received by the analysis platform 1912 and (ii) the destinations to which data is transmitted by the analysis platform 1912. Assume, for example, that the analysis platform 1912 resides on a computer server. In such embodiments, the communication module 1908 can communicate with sources 1910A-N external to the computing device 1900 from which to obtain RNA seq data and associated metadata. This metadata may include physiological information, treatment information, contextual information, or any combination thereof. Examples of physiological information include age, gender, height, weight, disease classification, disease outcome, and the like. Examples of treatment information include medications and corresponding regimens, surgical procedures, chemotherapy procedures, and the like. Examples of contextual information include geographical location, name and location of healthcare professional that rendered service, name and location of healthcare facility where service was rendered, and the like. Physiological information, treatment information, and contextual information could be acquired by the analysis platform 1912 from one of the sources 1910A-N, derived by the analysis platform 1912 (e.g., via analysis of metadata that accompanies the RNA seq data), provided by patients (e.g., via a survey or other mechanism), or specified by healthcare professionals (e.g., by allowing access to electronic health records).

For convenience, the analysis platform 1912 is referred to as a computer program that resides within the memory 1904. However, the analysis platform 1912 could be comprised of software, firmware, or hardware that is implemented in, or accessible to, the computing device 2100. In accordance with embodiments described herein, the analysis platform 1912 can include a processing module 1914, visualization module 1916, analysis module 1918, and graphical user interface (“GUI”) module 1920. These modules could be integral parts of the analysis platform 1912, or these modules could be logically separate from the analysis platform 1912 but operate “alongside” it. Together, these modules enable the analysis platform 1912 to produce visualizations that allow for insights into the relationships between different patients with brain tumors, as well as the implications of having a given disease/classification/mutation (e.g., either tumor or germline). For instance, the GUI 1920 may include one or more touch-sensitive displays that may be utilized for visualization and/or exploration (e.g., pinch-zoom, finger-as-cursor to encircle a cluster, writing-to-text data entry, menu operation, or color selection).

The processing module 1914 can process data that is obtained by the analysis platform 1912 into a format that is suitable for the other modules. For example, the processing module 1914 can apply operations to RNA seq data acquired from the sources 1910A-N in preparation for analysis. For example, the processing module 1914 can filter or alter the RNA seq data (e.g., to address batch effect), such that the RNA seq data can be more readily analyzed. As another example, the processing module 1914 may parse multiple datasets acquired from different sources and then combine the multiple datasets into a single dataset comprised of RNA seq data from the different sources. Such an approach may be helpful in ensuring that RNA seq data acquired from more than one source is being used consistently and similarly by the analysis platform 1914.

As discussed above, the analysis platform 1912 can produce various visualizations based on the RNA seq data. The visualization module 1916 may be responsible for producing these visualizations, which are discussed at length above. Moreover, the visualization module 1916 may be responsible for implementing the approaches set forth below. The analysis module 1918 may be responsible for computing metrics based on these visualizations or underlying RNA seq data. For example, the analysis module 1918 may be responsible for identifying nearest neighbors for a patient that is newly added to the visualization as further discussed below.

Meanwhile, the GUI module 1920 may be responsible for either causing display of visualizations produced by the visualization module 1916 on the display 1906 or generating a message (e.g., comprised of data packets) that is provided to the communication module 1908 for transmittal to a destination. The destination could be another computing device (e.g., a mobile phone associated with a patient to whom feedback is intended to be surfaced).

FIG. 9 shows a block diagram illustrating an example of a processing system 3000 in which at least some operations described herein can be implemented. For example, components of the processing system 3000 may be hosted on a computing device that includes an analysis platform (e.g., analysis platform 1702 of FIG. 6 or analysis platform 1912 of FIG. 8).

The processing system 3000 can include a processor 3002, main memory 3006, non-volatile memory 3010, network adapter 3012, video display 3018, input/output devices 3020 (e.g., a touchscreen may provide both video display and I/O functions), control device 3022 (e.g., a keyboard or pointing device such as a computer mouse or trackpad), drive unit 3024 including a storage medium 3026, and signal generation device 3030 that are communicatively connected to a bus 3016. The bus 3016 is illustrated as an abstraction that represents one or more physical buses or point-to-point connections that are connected by appropriate bridges, adapters, or controllers. The bus 3016, therefore, can include a system bus, a Peripheral Component Interconnect (“PCI”) bus or PCI-Express bus, a HyperTransport (“HT”) bus, an Industry Standard Architecture (“ISA”) bus, a Small Computer System Interface (“SCSI”) bus, a Universal Serial Bus (“USB”) data interface, an Inter-Integrated Circuit (“I2C”) bus, or a high-performance serial bus developed in accordance with IEEE 1394.

While the main memory 3006, non-volatile memory 3010, and storage medium 3026 are shown to be a single medium, the terms “machine-readable medium” and “storage medium” should be taken to include a single medium or multiple media (e.g., a centralized/distributed database and/or associated caches and servers) that store one or more sets of instructions 3028. The terms “machine-readable medium” and “storage medium” shall also be taken to include any medium that is capable of storing, encoding, or carrying a set of instructions for execution by the processing system 3000.

In general, the routines executed to implement the embodiments of the disclosure can be implemented as part of an operating system or a specific application, component, program, object, module, or sequence of instructions (collectively referred to as “computer programs”). The computer programs typically comprise one or more instructions (e.g., instructions 3004, 3008, 3028) set at various times in various memory and storage devices in a computing device. When read and executed by the processors 3002, the instruction(s) cause the processing system 3000 to perform operations to execute elements involving the various aspects of the present disclosure.

Certain embodiments are directed to virtual reality implementations, in which the visualization is done via a 2D or 3D with a “VR” headset, and the GUI can include simply moving one's head/gaze and also using a variety of gesture or physical hand-controller input mechanisms.

Further examples of machine-and computer-readable media include recordable-type media, such as volatile memory devices and non-volatile memory devices 3010, flash-drives, removable disks, hard disk drives, and optical disks (e.g., Compact Disk Read-Only Memory (“CD-ROMs”) and Digital Versatile Disks (“DVDs”)), and transmission-type media, such as digital and analog communication links.

The network adapter 3012 enables the processing system 3000 to mediate data in a network 3014 with an entity that is external to the processing system 3000 through any communication protocol supported by the processing system 3000 and the external entity. The network adapter 3012 can include a network adaptor card, a wireless network interface card, a router, an access point, a wireless router, a switch, a multilayer switch, a protocol converter, a gateway, a bridge, bridge router, a hub, a digital media receiver, a repeater, or any combination thereof.

Various aspects of the disclosure are directed to providing technical advantages that involve practical and tangible outcomes and uses including one or more of the following: an actual medical diagnosis (e.g., a specific type of cancer) generated from one of more of the computer-implemented examples disclosed herein and in which the diagnosis has a likelihood of certainty (e.g., as a statistically-based percentage or via weighting criteria relative to another set of patients used in the method), and such likelihood of certainty may be based on the most specific or last iteration of calculated centroid values relative to target patients. As other such technical advantage, certain examples may involve using output data and/or generated data corresponding to a diagnosis or a way to treat a patient (e.g., to be validated and approved by a medical professional), such as by: establishing a treatment plan; generating a prescription; ordering a medical procedure or test (e.g., radioactive imaging); and/or starting, modifying or stopping a specific treatment. Another such technical advantage example involves robotic surgery and/or surgical procedure leveraging from such exemplary methods disclosed herein, such as biopsies which may be modified in real time based on assessment of tissue (e.g., to assess margin area around a cancerous tumor) and/or other characteristics of the patient being operated on and approaches herein for assessing target patients.

Many different types of processes and devices in which a landscape of RNA expression profiles may be generated from a plurality of patients having a medical diagnosis that is common to the patients, and thereafter utilized to land a target patient in a group of the plurality of patients having corresponding RNA expression profiles. Various aspects may be advantaged by such aspects, the above aspects and examples as well as others (including the related examples in the '717 Provisional. The following characterizes various embodiments in the context of the figures in the '717 Provisional.

An example of a visualization as may be utilized in connection with various embodiments is provided in FIG. 1. Specifically, FIG. 1 includes a two-dimensional or three-dimensional visualization (also called a “landscape”) of data that is obtained from five sources, namely, the Genotype-Tissue Expression (“GTEx”) database, The Cancer Genome Atlas Glioblastoma Multiforme (“TCGA-GBM”) data collection, The Cancer Genome Atlas Low Grade Glioma (“TCGA-LGG”) data collection, the Chinese Glioma Genome Atlas (“CGGA”) database, and the Children's Brain Tumor Network (“CBTN”) database. Across these five sources, the analysis platform may obtain hundreds or thousands of samples.

Focusing on adult gliomas, the visualization illustrates that the corresponding individuals are largely, if not entirely, documented in two datasets, namely, TCGA-GBM and TCGA-LGG as shown in FIG. 2A. FIG. 2B illustrates the adult gliomas colored by common glioma alterations, including age at diagnosis, gain of chromosome 7 and loss of chromosome 10, codeletion 1p and 19q marks, mutation in the IDH1 gene, mutation in the TP53 gene, and mutation in the ATRX gene. FIG. 2C, meanwhile, illustrates overlapping of the CGGA dataset onto the TCGA-GBM and TCGA-LGG datasets. In the top row, the CGGA dataset is represented using gray visual indicia, and in the bottom row, the TCGA-GBM and TCGA-LGG datasets are represented using gray visual indicia.

To gain a better understanding of brain tumors more generally, the analysis platform can recast the UMAP model with only the data relating to adult and pediatric brain tumors. FIG. 3 illustrates how recasting enables a user of the analysis platform to better understand the distribution of, and relationship between, different types of brain tumors. Here, for example, 26 different brain tumors are represented in the recasted UMAP model. With this recasted UMAP model, the analysis platform can also gain insights into the brain tumors of the underlying patients. FIG. 4A illustrates how the adult and pediatric brain tumors can be segmented-for example, using a visual aid such as color, intensity, pattern, and the like-by gene expression or mutation. Such a visualization may enable an operator (also called a “user”) to better understand how these adult and pediatric brain tumors develop. FIG. 4B, meanwhile, is representative of another version of the visualization shown in FIG. 4A with normal brain tissue added and color used to show pathway activation. Again, such a visualization may enable a user to better understand development of these adult and pediatric brain tumors, particularly in conjunction with an analysis of normal brain tissue.

The analysis platform could also recast the UMAP model with a different subset of data. FIG. 5A, for example, illustrates the distribution of known chromosomal gains and losses in adult gliomas. In viewing the visualization of FIG. 5A, a user may be able to better understand the relative frequency and proximity of losses and gains relating to chromosomes 1p, 7p, 19q, and 10p. As mentioned above, the proximity between two points in one of these visualizations may be indicative of the similarity in the underlying data. FIG. 5B, meanwhile, illustrates the distribution and frequency of mutations and the distribution and frequency of gene fusions for adult brain tumors.

Through analysis of visualizations, the analysis platform can also compute, derive, or otherwise establish insights into different subtypes of brain tumors. Assume, for example, that the analysis platform is tasked with determining differences between different subtypes of medulloblastoma or establishing the relationship between a new patient diagnosed with medulloblastoma and existing patients known to have different subtypes of medulloblastoma. In such a scenario, the analysis platform could recast the UMAP model based on the data associated with existing patients known to have medulloblastoma. FIG. 6A illustrates the distribution of patients known to have medulloblastoma amongst five different classifications, namely, Grade 3 Tumor, Grade 4 Tumor, Sonic Hedgehog (“SHH”), Wingless Activated (“WNT”), and To Be Classified. These classifications may be established based on an analysis of data associated with the corresponding patients or metadata that accompanies the data (such as classification by a pathologist with a microscope). By noting the classifications of nearby patients, the analysis platform can make an inference about one or more neighbor patients. Additionally, by highlighting patients by their given diagnosis, placement in a cluster of patients of a different diagnosis can indicate that the medical records contain a diagnosis error, which is a valuable finding.

In FIG. 6A, the gray points correspond to patients that are diagnosed as having another brain tumor (i.e., not medulloblastoma). FIG. 6B shows how some patients diagnosed with medulloblastomas landed in the region generally corresponding to embryonal tumors and atypical teratoid rhabdoid tumors (“ATRTs”), while FIG. 6C shows how some patients diagnosed with embryonal tumors landed among the patients diagnosed with medulloblastomas. Where errors are made, for example where pathologists make (erroneous) subjective diagnoses between visually similar samples, such errors may be identified in response to patients being placed in a cluster that does not correspond to an indicated diagnosis.

### Interactive Visualizations for Understanding Relationships Between Patients

To better understand the functionality of the analysis platform and its visualizations, embodiments are discussed below in the context of meningiomas. However, those skilled in the art will recognize that the features of these embodiments may be equally applicable to other types of brain tumors and diseases more generally. Accordingly, while embodiments may be described in the context of meningiomas for the purpose of illustration, those skilled in the art will recognize that the features of those embodiments may be similarly applicable to other cancers and diseases more generally.

To understand the effects of meningiomas, it helps to understand how meningiomas develop. While meningiomas are generally thought to be benign, meningiomas can reoccur and invade other tissues, potentially spreading to other parts of the body. FIG. 7 shows how, through visual analysis of tumor cells under a microscope, meningiomas are generally characterized as Grade 1, Grade 2, or Grade 3. Grade 1 covers benign meningiomas that grow slowly and have distinct borders. Grade 3 covers malignant meningiomas that grow quickly and tend to aggressively invade nearby parts of the brain. Grade 2 covers atypical meningiomas that comprise tumor cells that do not appear typical or normal. These atypical meningiomas are generally not initially categorized as benign (Grade 1) or malignant (Grade 3) but may become malignant at some point. With roughly 25 percent recurrence in five years, it is important for meningiomas—especially atypical and malignant ones—to be quickly screened and addressed as prognosis improves with earlier detection and treatment.

Recurrent mutations have been identified in sporadic meningiomas. As shown in FIG. 8, the most common recurrent mutation is NF2 gene loss while the remaining recurrent mutations tend to relate to the TRAF7 gene, AKT1 gene, KLF4 gene, or SMO protein. In predicting onset or progression of meningiomas, several questions tend to be the focus of research. These questions include “Which patient is likely to have a tumor recurrence?” and “What is the biology of the brain tumors likely to recur?” Some research has focused on answering these questions via analysis with methylation, copy number, or gene mutation. However, such research has been inconclusive, and therefore, the analysis platform may instead attempt to answer these questions using sequencing information, for example, in the form of RNA seq data.

### A. Introduction to Demonstrating Differences Between Patients

Meningiomas are the most common intracranial tumor in humans. While most of these tumors are benign, some of these tumors are malignant, rapidly recur after intervention (e.g., surgery), and are ultimately lethal. The histologic grading employed by the World Health Organization (“WHO”) classification system identifies many of these meningiomas, but some meningiomas that are identified as Grade I or Grade II are equally aggressive as those identified as Grade III. Accordingly, better characterizations of the biology of aggressive meningiomas are needed. While several classification systems based on DNA methylation, copy number, or expression signatures have been proposed, these classification systems fail to fully address the drawbacks of relying entirely on the WHO classification system.

Clues to the underlying biology of meningiomas can be found through systematic analysis of the NF2 gene, as loss of the NF2 gene has not only been shown to be a common basis for spontaneous meningiomas but is also associated with the majority of rapidly recurrent meningiomas. The NF2 gene, which encodes the protein merlin, is a tumor suppressor that regulates the YAP1 protein (or simply “YAP1”) via the Hippo signaling pathway (or simply “Hippo pathway”). Upon contact inhibition, the Hippo pathway phosphorylates YAP1 resulting in the inhibition of YAP1 activity. In the absence of merlin, YAP1 remains active and translocates into the nucleus, binding the transcriptional enhanced associate domain (“TEAD”) transcription factors and activating cell proliferation. In addition to loss of NF2 gene function by chromosome 22 loss, meningiomas also lose NF2 gene function by inactivating point mutations and gene fusions, resulting in constitutively active YAP1 that is insensitive to Hippo pathway inactivation. Modeling experiments in mice have shown that the expression of either constitutively active YAP1 or YAP1 gene fusions found in human meningiomas induce similar tumors in mice.

Available therapeutic options for individuals with aggressive meningiomas are limited to radiation and multiple surgeries—which carry significant risk—and therefore a better understanding of the underlying biology of aggressive meningiomas is needed. It is likely that rapid recurrence and aggressive behavior of some meningiomas reflects that tumor's underlying biology, which is in turn reflected by its overall gene expression pattern. In the hope of understanding this aggressive subset of meningiomas and being able to predict which meningiomas are, or will become, aggressive, the analysis platform was tasked with performing an analysis of RNA seq data associated with individuals known to have different types of meningiomas. As further discussed below, the biology of different types of meningiomas could then be defined based on the RNA seq data and insights gleaned through the analysis.

Using the expression levels of all protein-coding genes, the analysis platform created a reference landscape (also called a “reference representation” or “reference map”) of about 1,300 tumors with associated metadata. FIG. 9 includes an example of a reference landscape created by applying a graph layout algorithm to RNA seq data acquired from 13 different sources. In this instance, the reference landscape is created using the UMAP algorithm, and therefore the reference landscape could also be called a “UMAP visualization” or “UMAP representation.” In the reference landscape, each visual indicium is representative of a different patient and its location is based on the corresponding RNA seq data.

The analysis platform can uncover relationships between different patients with meningiomas—and the relationship between a newly diagnosed patient and existing patients known to have meningiomas—through analysis of visualizations like the reference landscape shown in FIG. 9. For example, in reviewing the reference landscape shown in FIG. 9, the analysis platform discovered that there are at least nine meningioma subtypes, some of which are associated with distinct characteristics (e.g., time to recurrence), that can be distinguished from each other by gene expression similarities to developmental cell types and biological pathways. Moreover, the analysis platform determined that several subtypes were associated with particularly poor outcomes, the most aggressive of these subtypes exhibited high proliferation rates and RNA expression resembling muscle development. These insights are further discussed below.

To make insights gleaned from analysis of the reference landscape more translatable, the analysis platform can map newly diagnosed patients onto the reference landscape and, based on the locations of those newly diagnosed patients, predict tumor behavior, outcome, etc. As an example, predictions can be made for a newly diagnosed patient based on the characteristics of the nearest neighboring tumors (and corresponding existing patients) in the reference landscape. This reference map can be highly beneficial in clinical situations to predict outcomes and determine appropriate therapeutic strategies. As further discussed below, the analysis platform can not only create helpful visualizations but may also allow for interactive and analytical exploration of tumors (and corresponding patients) along with various associated metadata.

### B. Illustrative Examination of Process for Constructing the Reference Landscape

Initially, the analysis platform acquired 12 datasets comprised of RNA seq data from 9 institutions and 5 countries in North America, Europe, and Asia, and then combined with 279 sequenced meningiomas from the University of Washington to create a set of about 1,300 meningiomas. The analysis platform collected nucleotide base sequences from FASTQ files in each dataset and aligned the nucleotide base sequences to the human reference genome (i.e., HG38) using the “pipeline” shown in FIG. 10. The term “pipeline” may be used to refer to a series of software instruction sets, algorithms, or modules that are executed in sequential order, generally with the output of each being suppled as input to the next. To remove batch effects from different datasets, the analysis platform employed ComBat-seq, a batch effect adjustment tool for bulk RNA seq data, from the surrogate variable analysis (“sva”) package for the R software environment. The sva package includes functions for identifying and building surrogate variables for high-dimensional datasets. When employed, the sva package can be used to remove artifacts in three ways, namely, (i) identifying and estimating surrogate variables for unknown sources of variation in high-throughput experiments, (ii) directly removing known batch effects using ComBat, and (iii) removing batch effects with known control probes.

The analysis platform then normalized gene expression values from the datasets and converted to units of TPM. Thereafter, the analysis platform applied the UMAP algorithm on the batch-corrected, normalized TPM counts to create the reference landscape. As shown in FIG. 9, the reference landscape includes multiple clusters of different sizes, and these clusters are comprised of visual indicia corresponding to a mix of the 13 datasets with the exception of the HKU/UCSF dataset for which two small unique clusters were formed. For the 13 datasets, the UMAP algorithm better distinguished clusters that showed differences in clinical and genomic features compared to other dimensionality reduction algorithms, though a different dimensionality reduction algorithm may exhibit better results if the underlying data related to patients with a different type of cancer or an entirely different type of disease. The reference landscape not only facilitates two-and three-dimensional visualization of the data included in the 13 datasets but may also allow for interactive analysis.

To better understand characteristics of the underlying data included in the 13 datasets, the analysis platform created a series of visualizations that are shown in FIGS. 11A-J. Specifically, the analysis platform used a visual aid to differentiate visual indicia in the reference landscape from each other, generally based on the underlying RNA seq data or corresponding metadata. Here, color is used as the visual aid, though those skilled in the art will recognize that another visual aid, such as intensity or pattern, visibly differently-shaped markers, and changes in colors or animation (e.g., movement or color change) could be used to differentiate visual indicia from each other. Referring to FIGS. 11A-J, for any coloring scheme, the analysis platform only colored known values—tumors with no known values for a given characteristic were left uncolored. Further, color palates may be chosen so that those with color blindness conditions may properly view the presented data.

More than half of meningiomas—about 73 percent of tumors for which NF2 gene status is available—exhibit functional loss of the NF2 gene, which is achieved via the loss of chromosome 22, point mutations, or gene fusions. FIG. 11A includes a colored reference landscape showing tumors with and without known chromosome 22 loss, with one region being clearly highlighted. Point mutations and gene fusions leading to inactivation of the NF2 gene also tend to cluster with the tumors associated with chromosome 22 loss as shown in FIGS. 11B-C. FIG. 11B includes a colored reference landscape showing tumors with and without point mutations, and FIG. 11C includes a colored reference landscape showing tumors with and without gene fusions. Coloring the reference landscape for all three mechanisms of NF2 inactivation (i.e., chromosome 22 loss, point mutations, gene fusions) demonstrates a near complete loss of the NF2 gene across this region of the reference landscape, which is characterized by overall downregulated expression of the NF2 gene as shown in FIG. 11D. FIG. 11D includes a colored reference landscape showing expression of the NF2 gene.

NF2 wild-type YAP1 fusion-positive meningiomas also mapped onto the same region of the reference landscape, indicating that these meningiomas resemble NF2 mutant meningiomas on a gene expression level, as shown in FIG. 11E. Other recurrent non-NF2 mutations including to the TRAF7 gene and SMO gene were found distributed across the NF2-wildtype region of the reference landscape, while mutations to the KLF4 gene and AKT1 gene additionally showed high regionality for recurrent mutations as shown in FIG. 11F. The regionality of these genetic alterations is consistent with the known unique biology of meningiomas. For example, meningiomas that harbor mutations in the TRAF7 gene and KLF4 gene are predominantly regionalized to a single cluster.

Most of the samples in the 13 datasets are associated with a WHO grade, and coloring the reference landscape based on those WHO grades shows a nonrandom distribution. FIG. 11G includes a colored reference landscape showing distribution among WHO Grade I, Grade II, and Grade III. As shown in FIG. 11G, a subset of the region characterized by loss of the NF2 gene has an increased concentration of WHO Grade II and Grade III tumors relative to the remainder of the reference landscape. However, the region with the highest concentration of WHO Grade II and Grade III tumors still contained tumors of all three WHO grades. For some tumors, the underlying data indicated whether the sample was a first resection or a recurrent resection. FIG. 11H includes a colored reference landscape showing that tumors known to be recurrent at the time of resection are generally also concentrated in the same region as tumors with a higher WHO grade. For some tumors, the underlying data also indicated the time between the surgery in which the sample was generated and the next resection or previous resection. FIG. 11I includes a colored reference landscape showing that a short time to recurrence was enriched in that same region as the higher average WHO grade and increased likelihood of being a recurrent tumor.

Nearly all of the samples in the 13 datasets include an indication of age and gender of the corresponding patient. Consistent with what is known, the majority of the reference landscape comprised older patients that were predominantly female, about 66 percent female with a median age of about 58 years. FIG. 11J includes a colored reference landscape showing distribution of samples by gender, while FIG. 11K includes a colored reference landscape showing distribution of samples by age. There are two regions of the reference landscape that varied from this general rule. One region in which the patients were largely male, with about 61 percent male versus about 31 percent male in the rest of the reference landscape, and another adjacent region that includes a higher percentage of younger patients than the general population reflected in the reference landscape, with about 22 percent below 30 years old versus about 5 percent below 30 years old in the rest of the reference landscape.

### B. Alternative Classification Systems Also Exhibit Regional Patterns

Because the WHO classification system does not identify all meningiomas with aggressive behavior, several alternative classification systems have been proposed that rely on methylation patterns, copy number alterations, and gene expression to place patients into specific groups associated with different times to recurrence. FIGS. 12A-C include reference landscapes colored using the metadata of classification systems based on (i) RNA classification of NF2 wild-type benign, NF2 loss intermediate, and NF2 loss malignant; (ii) DNA methylation-based classification, and (iii) methylation profile classification. Additionally, the expression of the 34 genes presented as a signature to predict meningioma outcome was analyzed by the analysis platform in correlation to the reference landscape. Enriched and suppressed genes in the most aggressive meningiomas were divided into two gene sets, and the entire dataset was subjected to Gene Set Variation Analysis (“GSVA”) using the two gene sets separately. After obtaining two sets of GSVA scores, the analysis platform computed a ratio and then used the ratio to color the reference landscape to visually highlight the most aggressive region as shown in FIGS. 12D-F. Accordingly, even these alternative classification systems show regional patterns across the reference landscape.

### C. Identifying Meningioma Subtypes, Some With Distinct Characteristics

One of the benefits of the reference landscape is that its dimensional reduction, performed by the dimensionality reduction algorithm (e.g., the UMAP algorithm), and overlaying of known mutational and clinical metadata highlighted several potential meningioma subtypes. The analysis platform can use a clustering algorithm, such as Density-Based Spatial Clustering of Applications with Noise (“DBSCAN”), on either the 2D or 3D coordinates of the visual indicia to define specific clusters with statistical confidence. FIG. 13A illustrates how distinct clusters can be identified through statistical analysis of the spatial relationships between the visual indicia. Here, the analysis platform identified nine clusters that have been labeled A through H. The region with functional loss of the NF2 corresponds to clusters A and B. Cluster A includes the highest density of aggressive tumors, and cluster B represents the remainder of the NF2 gene loss region of the reference landscape with relatively benign tumors. Clusters C and D included mostly NF2 wild-type tumors. The comparison of Kaplan-Meier plots of time to recurrence identified clusters A and G as having the shortest time to recurrence, as shown in FIG. 13B. Patients in cluster C were found to experience significantly worse outcomes than patients in clusters B, D, E, and F, all of which were similar. The analysis platform refrained from analyzing clusters H and I due to the small number of patients in those clusters.

Some characteristics not only varied between clusters, but also within clusters. For example, regional differences in time to recurrence within a cluster was observed for many of the clusters. The most striking differences were observed in clusters A and C. The analysis platform was able to identify three subclusters—namely, A1, A2, and A3—within cluster A based on the regionalization of the most aggressive tumors and differences in patient outcome, as shown in FIG. 13C. FIG. 13D includes a Kaplan-Meier plot showing the recurrence rate for the three subclusters of cluster A. Subcluster A3 included the largest population of patients with poor outcomes, while subcluster A2 demonstrated the worst clinical outcomes, though it only contains 8 samples. Subclusters A2 and A3 were also highlighted as the most aggressive areas in FIG. 12F. Cluster C can similarly be divided into four subclusters—namely, C1, C2, C3, and C4—with significant differences in outcome, as shown in FIG. 13E. FIG. 13F includes a Kaplan-Meier plot showing the recurrence rate for the four subclusters of cluster C. Subcluster C2, like subcluster A2, only contains 8 samples but corresponds to a short time to recurrence. Several of the other clusters can also be subdivided into regions with difference differences in characteristics (e.g., outcome, time to recurrence, etc.). However, these other clusters generally represent less aggressive tumor types with few recurrences in general and longer times to recurrence.

Additional information on differences in characteristics across different clusters and subclusters can be found in Appendix A of the '717 Provisional.

### D. Biological Significance of Meningioma Subtypes

As mentioned above, the analysis platform created the reference landscape using RNA seq data, and therefore it presents a significant advantage in terms of performing differential gene expression analysis and deciphering the underlying biology across (and within) different meningioma subtypes. Initially, the analysis platform determined the differentially expressed genes in each cluster relative to the rest of the meningiomas. Then, the analysis platform performed gene ontology (“GO”) analysis. The most prevalent GO terms in each cluster were used to discern the underlying biological signature for each cluster as shown in FIGS. 14A-C. FIG. 14A includes a visualization of GSVA scores across the reference landscape for selected GO terms. In FIG. 14A, a score closer to +1 suggests upregulation of the respective gene set while a score closer to −1 suggests downregulation of the respective gene set. FIG. 14B shows the top 15 GO terms enriched in clusters A and C, while FIG. 14C shows a summary of the biological significance of each cluster. From FIGS. 14A-C, insights can be gleaned into the different clusters. For example, cluster A is enriched for cell cycle, skeletal and cardiac muscle development, and DNA replication and repair while cluster B is enriched for immune cells and function. Although cluster C has relatively fewer GO terms that do not point towards a specific biological signature, SMO mutations are enriched in cluster C. Accordingly, regulation of smoothened signaling and SHH pathway were upregulated within cluster C. Similarly, cluster D has a broad collection of GO terms, but is enriched for AKT1 mutations. Clusters E and F are enriched for epidermis development and vascular development, respectively. It is worth noting that KLF4—a transcription factor involved in skin development—was highly mutated in tumors in cluster D, specifically the K409Q mutation. Cluster G, which is associated with some of the worst outcomes, is enriched for neuronal functions including neurotransmitter/synaptic transmission and nervous system development. Clusters H and I are enriched for protein translation, macromolecule biosynthesis, and mitochondrial functions. Again, because clusters H and I include a small number of samples, any insights gleaned by the analysis platform form analysis of these samples may be considered tentative and used as a hypothesis for study of a larger set of samples.

Some of these cluster-specific biological signatures are related to different developmental pathways and cell types. To further learn whether meningioma subtypes are linked to developmental cell types, the analysis platform compared the gene signatures identified for the clusters to mouse embryonic cell types. Specifically, the analysis platform leveraged the transcription profiles of a series of mouse embryonic developmental stages and hundreds of cell types put together by the Shendure lab and described by C. Qiu et al. in “A single-cell transcriptional timelapse of mouse embryonic development, from gastrula to pup.” FIG. 14D shows the embryonic cell types that are determined to be enriched in each cluster. In line with the suggested GO terms, the analysis platform discovered that cluster A was enriched specifically for muscle progenitor cells and cardiomyocytes, and cluster B was enriched for immune cells. Cluster C was enriched for neuronal cells, though cluster D was not enriched for any specific embryonic cell type. Clusters E (skin related) and F (vascular related) were enriched for epithelial and endothelial cells, respectively. Cluster G was enriched for various neuronal cells.

### E. Recurrent Tumors Generally Do Not Experience Intercluster Movement

In the multiple datasets acquired by the analysis platform, there were several instances where samples were resected from multiple tumors in the same patient. Three scenarios were identified that could account for this, namely, (i) recurred tumors, (ii) multiple individual tumors from different brain regions, and (iii) progressed tumors due to incomplete surgical resection. The analysis platform evaluated the locations of these tumors on the reference landscape to better understand how biology and outcome might differ with time.

Generally, recurrent tumors remained within the clusters in which they were originally found, and vectors between multiple tumors associated with the same patient do not point towards a more aggressive region of the map. FIG. 15A shows the evolution of multiple tumors (i.e., primary and recurred tumors) from six patients, overlaid on the reference landscape. In FIG. 15A, arrows are drawn from the first tumor to the second tumor for each patient.

Regardless of the time between recurrences, this result suggests that the recurred tumors'biology and outcome do not vastly differ from the initial tumor. Within the collection of tumors, there are several instances where the multiple tumors are located in different clusters. FIG. 15B shows the evolution of multiple tumors (i.e., primary and recurred tumors) from six patients, overlaid on the reference landscape. In FIG. 15B, the patients are distinguished by color and number while the tumors are also distinguished by number (e.g., pt1.1 and pt1.2 are representative of the first and second tumors of the first patient). For each patient shown in FIG. 15B, the primary and recurred tumors occurred in different brain regions. The majority of these patients are also associated with loss of the NF2 gene.

There were also several instances where the tumors progressed due to previous incomplete surgical resection. FIG. 15C shows the evolution of these tumors that were not completely resected for two patients. In FIG. 15C, the patients are distinguished by color and number while the tumors are also distinguished by number (e.g., pt1.1 and pt1.2 are representative of the first and second tumors of the first patient). Despite these tumors progressing, the recurred tumors are located within the same cluster as the primary tumor.

### F. Overlaying New Patients Onto the Reference Landscape

Based on the analysis set forth above, it has been shown that biology and outcomes of meningiomas are regionally located in the reference landscape. Accordingly, for a new patient, the nearest neighboring existing patients (also called the “nearest neighbors”) can serve as references from which the tumor biology of the new patient and likely outcome can be inferred. However, in order to make such inferences, the analysis platform must be able to reliably map new patients onto the reference landscape.

Set forth below is a placement method for determining the appropriate location within the reference landscape for a new patient. As further discussed below, the placement method can use a weighted, nearest neighbors approach that leverages an ensemble of landscapes. For the purpose of illustration, the steps taken to validate the placement method are set forth below.

Initially, the analysis platform pretrained 100 low-dimensional graph representations with different initializations on the 13 datasets described above. Here, the 13 datasets served as a “reference dataset,” though any suitable dataset or combination of datasets could serve as the reference dataset. The low-dimensional graph representations were created using the UMAP algorithm, and therefore may also be called “UMAP representations.” FIG. 16A includes two examples of UMAP representations produced by UMAP algorithms trained with different random states. The analysis platform then used each UMAP model to map a new patient to a distinct 2D embedding. FIG. 16B shows how a new patient can be mapped onto each UMAP representation using the UMAP models. The analysis platform then used the locations of the new patient in each UMAP representation to determine which samples—included in the reference dataset—are the 100 nearest neighbors of the new patient in each UMAP representation within a radius determined using cross-validation. FIG. 16C illustrates how the nearest neighbors can be identified in each UMAP representation subject to a radius that is determined by cross-validation.

Such an approach results in 100 sets of nearest neighbors from the reference dataset, where each set is associated with a corresponding one of the 100 UMAP representations. The analysis platform can use this information to determine how frequently each sample in the reference dataset is a nearest neighbor of the new patient. FIG. 16D includes an example of a data structure (here, a matrix) that documents how often each sample in a reference dataset of 1,298 samples is determined to be a nearest neighbor for the new patient. Using this frequency information and the coordinates of the samples in the reference landscape, the analysis platform can compute the centroid of the coordinates of the samples in the reference landscape weighted by the frequency with which the samples were nearest neighbors of the new patient. The analysis platform can use the centroid as the final location for placing a visual indicium that is representative of the new patient on the reference landscape. FIG. 16E illustrates how, given the reference landscape with samples colored by the frequency that each is a nearest neighbor of a new patient, the new patient can be placed at the centroid of the nearest neighbors weighted by frequency.

To establish the reliability of the placement method, the analysis platform can use cross-validation to assess how far samples in the reference dataset move when the samples are removed from the reference dataset and then mapped back onto the reference landscape. First, the analysis platform can consider the location of each sample in the reference landscape as ground truth. FIG. 16F shows the ground truth location of a given sample that can be considered during cross-validation of the placement method. Next, the analysis platform can iteratively remove each sample, retrain the UMAP models without that sample, and use the placement method to map each sample back onto the reference landscape. FIG. 16G shows how the centroid of nearest neighbors can be identified for the given sample identified in FIG. 16F and then used to establish the appropriate location for the given sample to be mapped onto the reference landscape. Thereafter, the analysis platform can compute the distance (e.g., the Euclidean distance) between the ground truth location and the predicted location. FIG. 16H shows how the location of the given sample as predicted based on the centroid of the nearest neighbors can be compared against the ground truth location.

Following this cross-validation process, it has been found that nearly all samples were mapped back onto the reference landscape within a small radius of their true locations. FIG. 16I includes a plot illustrating the distribution of distances between the ground truth placement of samples and the centroid-based placements of those samples during cross-validation. The analysis platform also confirmed the reliability of the placement method by evaluating its predictive power. Results of this cross-validation process showed that the placement method was able to predict patient cluster member accurately (AUC=0.98) for substance clusters (i.e., where total sample count n>20) by predicting the cluster most common in the samples around which the corresponding patient was placed.

Cross-validation results also demonstrated the prognostic utility of the reference landscape. To leverage a patient's location on the reference landscape, the analysis platform can assign a location-based grade to each sample in the reference dataset that corresponded to the WHO grade most common in that sample's nearest neighbors once remapped onto the reference landscape. Results indicate that the predicted location grade is a superior risk indicator compared to WHO grade within WHO Grade I and Grade II meningiomas and, to a lesser extent, WHO Grade III meningiomas.

In univariate (single-variable) statistical analyses, WHO Grade I meningiomas were separated into predicted location WHO Grade I, Grade II, and Grade III with dramatically different recurrence-free survival (HR=2.6, p=2e-06). Similarly, WHO Grade II meningiomas that were classified as predicted location WHO Grade I had drastically better recurrence-free survival (HR=2.3, p=5e-05) compared to meningiomas classified as predicted location WHO Grade II and WHO Grade III. FIG. 16J includes Kaplan-Meier curves for predicted location within WHO Grade I, Grade II, and Grade III meningiomas in the reference dataset.

Although WHO Grade III meningiomas classified as predicted location WHO Grade I or Grade II may have more favorable outcomes than those predicted to be predicted location WHO Grade III (HR=2.7, p=0.02), all WHO Grade III meningiomas may experience short times to recurrence. Accordingly, despite the prognostic power of the reference landscape, histopathology plays a crucial role in assessing patient risk. Overall, the reference landscape is predictive of biology and outcome, and the ability of the analysis platform to place new patients on the reference landscape makes findings relevant for clinical applications.

Further information on the placement method, as well as the cross-validation process, can be found in Appendix A of the '717 Provisional. Various interfaces and use opportunities may be implemented, with the embodiments characterized herein (including those with FIGS. 1-5 of the '717 Provisional as discussed above).

As an illustrative example involving an analysis platform as characterized above, assume that a user of the analysis platform is interested in gaining greater insight into a patient cohort. FIG. 25 includes an interface with a visualization of patients for which clinical data (e.g., RNA seq data) is obtained from one of five databases. These databases include the TCGA-LGG data collection, TCGA-GBM data collection, GTEx database, CGGA database, and CBTN database. In FIG. 25, each patient is represented as a different digital element, allowing users to interact with the patient cohort in a more meaningful manner. Here, for example, a user selected the GTEx database in the pane along the left side of the interface. Upon receiving input that is indicative of the selection of the GTEx database, the analysis platform may emphasize the patients for whom clinical data was obtained from the GTEx database. Here, for example, the digital elements corresponding to those patients are outlined.

The user may also be permitted to hide patients whose clinical data was obtained from a certain database, whose clinical data satisfy a certain criterion, whose expression level for a certain gene falls outside a given range, etc. Referring to FIG. 26, for example, the user engaged the hide icon (here, an eye) that is shown in the pane along the left side of the interface. Upon receiving input that is indicative of the engagement, the analysis platform may deemphasize the digital elements of those elements or hide them entirely.

As mentioned above, the analysis platform may allow the user to create and save patient cohorts. Referring to FIG. 27, for example, the user encircled patients to be defined as a cohort. The visualization may be readily viewable in three dimensions or two dimensions, allowing for more precise encircling of patients (and therefore, defining of cohorts). Upon receiving input that is indicative of an identification of the patients to be included in a cohort, the analysis platform can perform several steps. First, the analysis platform may visually indicate which databases include clinical data of the encircled patients. Here, for example, the TCGA-LGG database and CGGA database are visually emphasized (e.g., bolded) in the pane along the left side of the interface. Second, the analysis platform may permit the user to name and save the cohort, so as to allow for easier retrieval (e.g., for comparison) in the future. Third, the analysis platform may generate one or more diagrams that summarize the clinical data associated with the encircled patients. Here, for example, the analysis platform has generated a survival plot showing the survival of the encircled patients in comparison to all patients, and the analysis platform has generated a copy number plot.

In FIG. 28, the user encircled more patients to be defined as a second cohort. While the content of the interface shown in FIG. 28 is comparable to the content of the interface shown in FIG. 27, the analysis platform could generate metrics and/or diagrams to illustrate the differences between the cohorts defined by the user. Here, for example, the analysis platform performs gene set variation analysis (“GSVA”) to produce metrics that indicate the differences between the cohorts, as determined through a comparison of clinical data. Moreover, the survival plot and copy number plot have been updated to account for the second cohort.

FIG. 29 includes another interface with a visualization of patients for which clinical data is obtained from one of five databases. Through this interface, the user has specified a signal of interest (here, sonic hedgehog—or “biocarta_shh_pathway”). Upon receiving input that is indicative of a selection or specification of a signal of interest, the analysis platform may update the visualization accordingly. For example, the analysis platform may visually emphasize digital elements corresponding to patients, if any, that have the signal of interest. Such an approach allows users to readily observe whether clustering naturally occurs for different signals (and, if not, whether the lack of clustering is useful in terms of developing appropriate plans of care).

It is recognized and appreciated that as specific examples, the above-characterized figures and discussion are provided to help illustrate certain aspects (and advantages in some instances) which may be used in the manufacture of such structures and devices. These structures and devices include the exemplary structures and devices described in connection with each of the figures as well as other devices, as each such described embodiment has one or more related aspects which may be modified and/or combined with the other such devices and examples as described hereinabove may also be found in the Appendices that form part of the above-referenced Provisional.

Other embodiments are directed to aspects disclosed in the '539 Provisional, including those noted in connection with FIG. 6A in what is referred to as “Appendix A” (as part of the '539 Provisional). Specifically, FIG. 6A depicts volcano plots showing differentially expressed genes in EPN-E1 (pink) and EPN-E2 (green), with differentially regulated kinases highlighted in blue, the top tyrosine kinase receptors are labeled in black. Such approaches may be utilized to assess the differently-expressed genes (e.g., with EPN-E1 and EPN-E2 encoding “epsins” (proteins) as may relate to endocytosis, in which EPN-E2 may have a more restricted expression relative to EPN-E1, and may have slightly different functional roles as can relate to cell adhesion and cancer development.

The skilled artisan would also recognize various terminology as used in the present disclosure by way of their plain meaning. As examples, the Specification may describe and/or illustrate aspects useful for implementing the examples by way of various modules/circuits which may be illustrated as or using terms such as layers, blocks, modules, device, system, unit, controller, and/or other circuit-type depictions. As other examples, “patient” may refer to a prospective or current medical patient with or without a specific diagnosis. Further reference to a noun in the singular may refer to one from among one or more of, unless otherwise indicated (e.g., “a patient” in various contexts is the same as referring to “at least one patient”), and reference to “example” is not intended to be limiting (e.g., “example” and “non-limiting example” are synonymous). Such aspects and circuit elements and/or related circuitry may be used together with other aspects to exemplify how certain examples may be carried out in the form or structures, steps, functions, operations, activities, etc. It should be understood that the terminology is used for notational convenience only and that in actual use the disclosed structures may be oriented and/or ordered different from the orientation or ordering shown in the figures. Thus, the terms should not be construed in a limiting manner.

Based upon the above discussion and illustrations, those skilled in the art will readily recognize that various modifications and changes may be made to the various embodiments without strictly following the exemplary embodiments and applications illustrated and described herein. For example, methods as exemplified in the Figures may involve steps carried out in various orders, with one or more aspects of the embodiments herein retained, or may involve fewer or more steps. Such modifications do not depart from the true spirit and scope of various aspects of the disclosure, including aspects set forth in the claims.

What is claimed:

1. A computer-executed method comprising:
generating a landscape of RNA expression profiles from a plurality of patients having a general medical diagnosis that is common to the patients; and
for a target patient having the general medical diagnosis and having a RNA expression profile,
identifying, from the landscape of RNA expression profiles, a plurality of refined nearest neighbor RNA expression profiles, wherein the plurality of refined nearest neighbor RNA expression profiles is based on: the target patient's RNA expression profile, one or more centroid values corresponding to the patients and being derived by use of filtering values that are associated with a likelihood that a computed distance from the one or more centroid values and that are to remove noisy nearest neighbors from the landscape not associated with patient-relationship insights drawn from placement of the target patient in a visualization of the landscape; and
identifying, for a trial involving a pharmaceutical prescribed to certain patients, the certain patients as corresponding to the plurality of refined nearest neighbor RNA expression profiles.

2. The computer-executed method of claim 1, further including the step of correlating outcomes or impact of the pharmaceutical as prescribed to the certain patients, as being positive or negative.

3. The computer-executed method of claim 1, further including the step of predicting, based on the landscape of RNA expression profiles, how the certain patients would be impacted by the pharmaceutical.

4. The computer-executed method of claim 1, wherein the plurality of refined nearest neighbor RNA expression profiles corresponds to a dimensionally-reduced data set derived by re-casting patient-profile data, corresponding to the RNA expression profiles from the plurality of patients, multiple times to lessen errors in the step of identifying the plurality of refined nearest neighbor RNA expression profiles.

5. The computer-executed method of claim 1, wherein the one or more centroid values are a function of samples in the landscape weighted by a frequency with which the samples were deemed to be within a set of nearest neighbors of one or more of the plurality of patients.

6. The computer-executed method of claim 1, wherein the plurality of refined nearest neighbor RNA expression profiles is further based on:
the one or more centroid values being derived from
calculating a centroid value, corresponding to one of or from among the one or more centroid values,
filtering to remove certain of the patients based on an adaptive distance from the centroid value to RNA expression profiles and therein identify a set of filtered patients;
recalculating the one or more centroid values, as a function of iterating sets of values, associated with the filtering and, in response, adjusting the certain patients identified as corresponding to the plurality of refined nearest neighbor RNA expression profiles.

7. The computer-executed method of claim 1, further including training a computer circuit, configured with a machine learning or artificial intelligence algorithm, to form or identify clusters, based on one or more additional RNA expression profiles landing on the landscape and on a changed set of patient characteristics used in the step of identifying the certain patients.

8. The computer-executed method of claim 7, wherein the changed set of patient characteristics is a function of a target patient's electronic medical record being associated with the one or more additional RNA expression profiles landing on the landscape.

9. The computer-executed method of claim 1, further including: calculating a centroid value, corresponding to one of or from among the one or more centroid values based on RNA expression profiles for the patients, and using the centroid value as a distance calculation; and filtering, based on characteristics associated with the RNA expression profiles, to adjust the distance calculation; and wherein the step of identifying the certain patients is at least partly based on the distance calculation being adjusted.

10. An apparatus comprising:
data-processing computer circuitry to generate a landscape of RNA expression profiles from a plurality of patients having a general medical diagnosis that is common to the patients;
for a target patient having the general medical diagnosis and having a RNA expression profile, using data-processing computer circuitry to
identify, from the landscape of RNA expression profiles, a plurality of refined nearest neighbor RNA expression profiles, wherein the plurality of refined nearest neighbor RNA expression profiles is based on: the target patient's RNA expression profile, one or more centroid values corresponding to the patients and being derived by use of filtering values that are associated with a likelihood that a computed distance from the one or more centroid values and that are to remove noisy nearest neighbors from the landscape not associated with patient-relationship insights drawn from placement of the target patient in a visualization of the landscape; and
identify, for a trial involving a pharmaceutical prescribed to certain patients, the certain patients as corresponding to the plurality of refined nearest neighbor RNA expression profiles.

11. The apparatus of claim 10, wherein the data-processing computer circuitry is to carry out a step of correlating outcomes or impact of the pharmaceutical as prescribed to the certain patients, as being positive or negative.

12. The apparatus of claim 10, wherein the data-processing computer circuitry is to carry out a step of predicting, based on the landscape of RNA expression profiles, how the certain patients would be impacted by the pharmaceutical.

13. The apparatus of claim 10, wherein the plurality of refined nearest neighbor RNA expression profiles corresponds to a dimensionally-reduced data set derived by re-casting patient-profile data, corresponding to the RNA expression profiles from the plurality of patients, multiple times to lessen errors in the step of identifying the plurality of refined nearest neighbor RNA expression profiles.

14. The apparatus of claim 10, wherein the one or more centroid values are a function of samples in the landscape weighted by a frequency with which the samples were deemed to be within a set of nearest neighbors of one or more of the plurality of patients.

15. The apparatus of claim 10, wherein the plurality of nearest neighbor RNA expression profiles is further based on:
the one or more centroid values being derived, by the data-processing computer circuitry
calculating a centroid value corresponding to one of or from among the one or more centroid values;
filtering to remove certain of the patients based on an adaptive distance from the centroid value to RNA expression profiles and therein identify a set of filtered patients; and
recalculating the one or more centroid values, as a function of iterating sets of values, associated with the filtering and, in response, adjusting the certain patients identified as corresponding to the plurality of refined nearest neighbor RNA expression profiles.

16. The apparatus of claim 10, wherein the data-processing computer circuitry is configured, via a machine learning or artificial intelligence algorithm being trained by repeated use of the data-processing computer circuitry to identify the plurality of refined nearest neighbor RNA expression profiles, to form or identify clusters based on one or more additional RNA expression profiles landing on the landscape and on a changed set of patient characteristics used in the step of identifying the certain patients.

17. The apparatus of claim 16, wherein the changed set of patient characteristics is a function of a target patient's electronic medical record being associated with the one or more additional RNA expression profiles landing on the landscape.

18. The apparatus of claim 10, wherein the data-processing computer circuitry is to carry out a step of calculating a centroid value, corresponding to one of or from among the one or more centroid values based on RNA expression profiles for the patients, and using the centroid value as a distance calculation; and filtering, based on characteristics associated with the RNA expression profiles, to adjust the distance calculation; and wherein the step of identifying the certain patients is at least partly based on the distance calculation being adjusted.

19. The apparatus of claim 10, wherein the data-processing computer circuitry is to carry out a step of training a computer circuit, configured with a machine learning or artificial intelligence algorithm, to form or identify clusters based on one or more additional RNA expression profiles landing on the landscape and on a changed set of patient characteristics used in the step of identifying the certain patients.
